(19) United States
(12) Patent Application Publication
(10) Pub. No.: US 2007/0065397 A1
(43) Pub. Date: Mar. 22, 2007

(54) Material having deodorizing function and process for production thereof

(76) Inventors: Hiroshi Ito, Tokyo (JP); Kazuya Akada, Tokyo (JP); Yukio Yasutake, Tokyo (JP)

Correspondence Address: OSTROLENK FABER GERB & SOFFEN, 1180 AVENUE OF THE AMERICAS, NEW YORK, NY, 100368403, US

(21) Appl. No.: 10/554,091

(22) PCT Filed: Apr. 22, 2004
(86) PCT No.: PCT/JP04/05763

§ 371 Date: Aug. 14, 2006

(30) Foreign Application Priority Data
Apr. 22, 2003 (JP) 2003-117573

(51) Int. Cl. A61L 9/00

(52) U.S. Cl. 424/76.2

(57) ABSTRACT

A material having a deodorizing function, characterized in that it comprises a paper powder containing a copper or zinc compound or copper and zinc compounds retained therein. A method for producing a material having a deodorizing function, characterized in that it comprises mixing an aqueous solution of a copper or zinc compound or copper and zinc compounds into a paper powder; mixing the resulting mixture into an aqueous solution of a pH adjuster to form a water-insoluble basic compound or hydroxide of copper or zinc or copper and zinc; and retaining the formed water-insoluble basic compound or hydroxide in the paper powder. It is possible to form a material having a deodorizing function by effectively utilizing a waste such a paper powder and to provide an inexpensive material having a deodorizing function and a method for producing the same. A granular excrement treating article comprising a granular core portion and a coating layer portion covering the granular core portion, both of which are formed utilizing the material having a deodorizing function. During and after use of the excrement treating article, an obnoxious odor generated by excrement can be deodorized by the copper or zinc compound having the deodorizing function.

### FIELD OF THE INVENTION

The present invention relates to a material having a deodorizing function and a process for producing the same, and more particularly, to an inexpensive material having sterilizing and deodorizing functions and general-purpose properties produced using a waste at least partially, and a process for producing the same. The material having the sterilizing and deodorizing functions according to the present invention can be formed, for example, into a powdery shape and used as a material having a deodorizing function for a core portion or a coating layer portion of an animal excrement treating article having the coating layer portion, or for an absorbing portion of a pet sheet, a paper diaper, a breast pad or a urine-absorbing pad. The material having the sterilizing and deodorizing functions according to the present invention can be also formed into a powdery shape and placed into a receptacle for use in a filter for a cooling/heating apparatus, a filtering medium for a bath hot water circulated within a bath tub, a boot insert, or can be formed into a suitable shape or placed into a receptacle for use for a refrigerator, a room interior, a toilet and the like. The material according to the present invention can be mixed into a paper material along with a sizing agent, a loading material and the like in a paper-making process for use in a wall paper, a shoji paper or the like. The present invention also relates to a granular excrement treating article having a deodorizing function for a human and/or an animal and produced using, as a starting material, particularly a material recovered from goods of life, or both of a material recovered from goods of life and a waste.

### BACKGROUND ART

A granular excrement treating article for an animal such as a domestic animal, a toy animal and the like is produced by using, as a starting material, an inorganic waste such as sand, bentonite, zeolite and the like, an organic waste such as a paper-making pulp and the like, or an organic waste such as a coffee-extraction residue, used tea leaves, a pulp sludge and the like, mixing a water-absorbable resin into the starting material and forming the resulting mixture into a small massive shape or a granular shape. Such a granular excrement treating article contains a deodorizer incorporated therein in order to odorize an odor of excrement after use of the article.

On the other hand, obnoxious odor components within a room include those based on nitrogen compounds such as ammonia and trimethyl amine, those based on sulfur compounds such as hydrogen sulfide, mercaptan and the like. Conventional materials having a deodorizing function and used to remove these odors include a material having an adsorbing function and a deodorizing function such as activated carbon, zeolite and the like capable of adsorbing and removing the odor components. However, when a deodorization is carried out using such a material an adsorbing function and a deodorizing function, the deodorizing ability of the material having the deodorizing function is reduced in the course of the deodorization continued. To restore the reduced deodorizing function, it is necessary to regenerate the material having the reduced deodorizing function, but the odor components once adsorbed are desorbed and released in the course of the regeneration to generate odors again, which is a problem. Therefore, as such a deodorizing material for removing odor components by a chemical reaction, a material having a deodorizing function and containing a complex, basic compound or hydroxide compound of copper, zinc or the like has been proposed (for example, see Japanese Patent No. 2557645).

In the material having the deodorizing function used in this Patent, however, it is required that a metallic compound having a deodorizing function such as silver nitrate, copper sulfate or zinc sulfate or a basic compound of silver, copper or zinc, or silver hydroxide, copper hydroxide or zinc hydroxide is carried on a cellulose fiber, so that it is prevented from being easily eluted or removed from the cellulose fiber. The metallic compound is firmly fixed to a cellulose fiber modified by a carboxymethyl group by reaction with this fiber. A firmly fixing means such as a fiber material such as a pulp and cellulose and carboxymethyl cellulose and another stabilizer is relatively expensive and hence, a material having a deodorizing function is also expensive, and the application thereof is limited due to the cost. This is a problem for providing a material having a general-purpose deodorizing function. Another problem is that when copper ion is bonded to carboxy-methylated cellulose, the generation of a reaction waste liquid a washing waste liquid is not avoided at a step of bonding the copper ion to the carboxy-methylated cellulose, and a large cost is required for the treatment of the waste liquid.

On the other and, a large amount of a paper diaper waste generated upon production of a paper diaper is pulverized and classified into a classification product containing mainly a pulp and a classification product containing mainly plastics, which are then recovered. However, the classification product containing the pulp has a small particle size and hence, the field of utilization of the classification product is limited. Moreover, even if the classification product is classified again, it is difficult to completely separate a water-absorbable resin. For this reason, the water-absorbable resin is not separated and is remained to exist in a state in which it has been mixed with the pulp. Thus, the application thereof is further limited, and confined to the use as a pulp for a paper diaper. However, as regards a fine pulp, it is difficult to even use it as a pulp for a paper diaper, which is a problem.

It is an object of the present invention to provide a granular excrement treating article at a low cost, in which a mixture of two or more of copper sulfate or zinc sulfate or a basic compound of copper or zinc, or copper hydroxide or zinc hydroxide or a metallic compound of copper or zinc is retained as a material having a deodorizing function. It is a further object of the present invention to provide a granular excrement treating article at a low cost, which has a deodorizing function and in which an operation for bonding a metallic compound having a deodorizing function to cellulose and a waste liquid treating operation associated with the bonding operation are not required. It is a yet further object of the present invention to solve the problems associated with the material cost of the conventional material having the deodorizing function.

### DISCLOSURE OF THE INVENTION

The present inventors have found that it is possible to provide a coated granulated matter having a deodorizing function, which is made by depositing or impregnating copper ion or the like having a deodorizing function onto or into a surface of a granulated material by spraying, and drying the resulting granulated material, so that a waste liquid cannot be generated, and a metallic compound having the deodorizing function such as a copper compound cannot be eluted during use, thereby accomplishing the present invention. The present inventors have also found that a paper powder having a relatively small particle size such as a pulp classification product containing a small amount of a water-absorbable resin can retain a complex, basic compound, hydroxide or other compound of copper or zinc, or complexes, basic compounds, hydroxides or other compounds of copper and zinc to produce a material having a relatively inexpensive deodorizing function, and particularly, relatively inexpensive sterilizing and deodorizing functions, thereby accomplishing the present invention.

It is another object of the present invention to provide a relative inexpensive material having a deodorizing function, which is produced by using, as a starting material, a classification product containing mainly a pulp and separated and recovered by classification from a reject article conventionally generated upon the production of sanitary goods such as a paper diaper or a waste of sanitary goods such as cutting wastage generated upon cutting, or using a reject article or cutting wastage of a thin paper sheet or a pulverized material of another wastepaper as a starting material, impregnating the pulverized material of the paper waste with a water-soluble copper or zinc compound or water-soluble copper and zinc compounds, water-insolubilizing the water-soluble compound of copper or zinc or the water-soluble compounds of copper and zinc and retaining it or them in a paper powder, thereby saving the material cost, and to provide a process for producing the same.

Namely, the present invention resides in a material having a deodorizing function, characterized in that it comprises a paper powder, and a copper compound or a zinc compound or a copper compound and a zinc compound contained and retained in the paper powder. The present invention also resides in a material having a deodorizing function, characterized in that it comprises a paper powder, carboxymethyl cellulose secured in the paper powder, and a copper or zinc compound or copper and zinc compounds contained and retained in the carboxymethyl cellulose. Further, the present invention resides in a material having a deodorizing function, characterized in that it comprises a paper powder, and a water-insoluble complex, basic compound or hydroxide compound of copper or zinc, or water-insoluble complexes, basic compounds or hydroxide compounds of copper and zinc retained in the paper powder. Yet further, the present invention resides in a material having a deodorizing function, characterized in that it comprises a paper powder, carboxymethyl cellulose secured in the paper powder, and a water-insoluble complex, basic compound or hydroxide compound of copper or zinc, or water-insoluble complexes, basic compounds or hydroxide compounds of copper and zinc contained and retained in the carboxymethyl cellulose. Further, the present invention resides in a granular excrement treating article characterized in that it is formed into a granulated material having a grain size equal to or larger than 1 mm and containing a powder of an organic waste, an amount of a material smaller than that of the powder of the organic waste and having an adhesive property, and a paper powder in which a copper compound or a zinc compound or a copper compound and a zinc compound each having a deodorizing function is or are retained.

Yet further, the present invention resides in a granular excrement treating article characterized in that it is formed into a granulated material having a grain size equal to or larger than 1 mm and containing a powder of an organic waste, an amount of a material smaller than that of the powder of the organic waste and having an adhesive property, an additive, and a paper powder in which a copper compound or a zinc compound or a copper compound and a zinc compound each having a deodorizing function is or are retained. Yet further, the present invention resides in a granular excrement treating article characterized in that it is formed into a granulated material having a grain size equal to or larger than 1 mm, and comprises a powder of an organic waste, an amount of a material smaller than that of the powder of the organic waste and having an adhesive property, and a paper powder with carboxymethyl cellulose secured therein for bonding or deposition of copper compound or a zinc compound or a copper compound and a zinc compound each having a deodorizing function. In addition, the present invention resides in a granular excrement treating article characterized in that it is formed into a granulated material having a grain size equal to or larger than 1 mm and containing a powder of an organic waste, an amount of a material smaller than that of the powder of the organic waste and having an adhesive property, an additive, and a paper powder with carboxymethyl cellulose secured therein for bonding or deposition of a copper compound or a zinc compound or a copper compound and a zinc compound each having a deodorizing function. Further, the present invention resides in a granular excrement treating article formed into a coated granulated material having a granular core portion and a coating layer portion covering the granular core portion, characterized in that the granular core portion is formed into a granular shape having a grain size equal to or larger than 1 mm, and contains a powder of an organic waste, an amount of a material smaller than that of the powder of the organic waste and having an adhesive property, and a paper powder in which a copper compound or a zinc compound or a copper compound and a zinc compound each having a deodorizing function is retained, and the coating layer portion is formed to cover at least a portion of the granular core portion, and contains a paper powder and a material having an adhesive property. Additionally, the present invention resides in a granular excrement treating article formed into a coated granulated material having a granular core portion and a coating layer portion covering the granular core portion, characterized in that the granular core portion is formed into a granular shape having a grain size equal to or larger than 1 mm, and contains a powder of an organic waste, an amount of a material smaller than that of the powder of the organic waste and having an adhesive property, an additive, and a paper powder in which a copper compound or a zinc compound or a copper compound and a zinc compound each having a deodorizing function is retained, and the coating layer portion is formed to cover at least a portion of the granular core portion, and contains a paper powder and a material having an adhesive property. Yet further, the present invention resides in a granular excrement treating article formed into a coated granulated material having a granular core portion and a coating layer portion covering the granular core portion, characterized in that the granular core portion is formed into a granular shape having a grain size equal to or larger than 1 mm, and contains a powder of an organic waste, and an amount of a material smaller than that of the powder of the organic waste and having an adhesive property; the coating layer portion is formed to cover the granular core portion, and contains a paper powder and a material having an adhesive property; and a paper powder retaining a copper compound or a zinc compound or a copper compound and a zinc compound each having a deodorizing function is contained in the granular core portion, between the granular core portion and the coating layer portion, in at least a portion of the coating layer portion or in at least a portion of a surface of the coating layer portion, or between the granular core portion and the coating layer portion, as well as in at least a portion of the coating layer portion or in at least a portion of a surface of the coating layer portion, or in at least a portion of the coating layer portion and in at least a portion of a surface of the coating layer portion. Yet additionally, the present invention resides in a granular excrement treating article formed into a coated granulated material having a granular core portion and a coating layer portion covering the granular core portion, characterized in that the granular core portion is formed into a granular shape having a grain size equal to or larger than 1 mm, and contains a powder of an organic waste, and an amount of a material smaller than that of the powder of the organic waste and having an adhesive property; the coating layer portion is formed to cover the granular core portion and contain a paper powder, a material having an adhesive property, and an additive; and an additive and a paper powder retaining a copper compound or a zinc compound or a copper compound and a zinc compound each having a deodorizing function are contained in the granular core portion, between the granular core portion and the coating layer portion, in at least a portion of the coating layer portion or in at least a portion of a surface of the coating layer portion, or between the granular core portion and the coating layer portion, as well as in at least a portion of the coating layer portion or in at least a portion of a surface of the coating layer portion, or in at least a portion of the coating layer portion and in at least a portion of a surface of the coating layer portion. Further, the present invention resides in a granular excrement treating article formed into a coated granulated material having a granular core portion and a coating layer portion covering the granular core portion, characterized in that the granular core portion is formed into a granular shape having a grain size equal to or larger than 1 mm, and contains a powder of an organic waste, an amount of a material smaller than that of the powder of the organic waste and having an adhesive property, and an additive; the coating layer portion is formed of a mixture of a paper powder and a material having an adhesive property to cover the granular core portion; and a paper powder retaining a copper compound or a zinc compound or a copper compound and a zinc compound each having a deodorizing function is contained between the granular core portion and the coating layer portion, in at least a portion of the coating layer portion or in at least a portion of a surface of the coating layer portion, or between the granular core portion and the coating layer portion, as well as in at least a portion of the coating layer portion or in at least a portion of a surface of the coating layer portion, or in at least a portion of the coating layer portion and in at least a portion of a surface of the coating layer portion. Further, the present invention resides in a granular excrement treating article formed into a coated granulated material having a granular core portion and a coating layer portion covering the granular core portion, characterized in that the granular.core portion is formed into a granular shape having a grain size equal to or larger than 1 mm, and contains a powder of an organic waste, an amount of a material smaller than that of the powder of the organic waste and having an adhesive property, and an additive; the coating layer portion is formed of a mixture of a paper powder and a material having an adhesive property to cover the granular core portion; and a paper powder with carboxymethyl cellulose secured therein for bonding or deposition of a copper compound or a zinc compound or a copper compound and a zinc compound each having a deodorizing function is contained between the granular core portion and the coating layer portion, in at least a portion of the coating layer portion or in at least a portion of a surface of the coating layer portion, or between the granular core portion and the coating layer portion, as well as in at least a portion of the coating layer portion or in at least a portion of a surface of the coating layer portion, or in at least a portion of the coating layer portion and in at least a portion of a surface of the coating layer portion.

In addition, the present invention resides in a process for producing a material having a deodorizing function, characterized by the step of mixing an aqueous solution of a copper compound or a zinc compound or a copper compound and a zinc compound into a paper powder to retain the copper compound or the zinc compound or the copper compound and the zinc compound in the paper powder. The present invention also resides in a process for producing a material having a deodorizing function, characterized by the steps of mixing an aqueous solution of a copper compound or a zinc compound or a copper compound and a zinc compound into a paper powder, mixing the resulting mixture into an aqueous solution of a pH adjuster to form a water-insoluble complex, basic compound or hydroxide of copper or zinc or copper and zinc, and allowing the formed water-insoluble basic compound or hydroxide of copper or zinc or copper and zinc to be retained in the paper powder. Further, the present invention resides in a process for producing a material having a deodorizing function, characterized by the steps of mixing an aqueous solution of a copper compound or a zinc compound or a copper compound and a zinc compound containing a citrate into a paper powder at a temperature equal to or higher than ambient temperature, mixing the resulting mixture into an aqueous solution of a pH adjuster to form a water-insoluble complex, basic compound or hydroxide of copper or zinc or copper and zinc, and allowing the formed water-insoluble basic compound or hydroxide of copper or zinc or copper and zinc to be retained in the paper powder. Yet further, the present invention resides in a process for producing a material having a deodorizing function, characterized by the steps of mixing an aqueous solution or suspension of a copper compound or a zinc compound or a copper compound and a zinc compound into carboxymethyl cellulose to adsorb or deposit ions of copper or zinc or copper and zinc on the carboxymethyl cellulose, and mixing the carboxymethyl cellulose with ions of copper or zinc or copper and zinc adsorbed thereon into a paper powder in the presence of water, thereby allowing the carboxymethyl cellulose with ions of copper or zinc or copper and zinc adsorbed thereon to be deposited on the paper powder.

In addition, the present invention resides in a process for producing a material having a deodorizing function according to any one of claims 24 to 27, characterized in that the hydroxide and basic compound of copper or zinc or copper and zinc retained in the paper powder is water-insoluble. Further, the present invention resides in a process for producing a granular excrement treating article, characterized by the steps of mixing, into a powder of an organic waste having a particle size equal to or smaller than 5 mm, an amount of a material smaller than that of the powder of the organic waste and having an adhesive property, granulating the resulting mixture to form a granulated matter having a grain size equal to or larger than 1 mm, allowing a copper compound or a zinc compound or a copper compound and a zinc compound each having a deodorizing function to be deposited on at least a portion of a surface of the formed granulated matter, thereby forming, as a core portion, a granulated matter having the copper compound or the zinc compound or the copper compound and the zinc compound deposited thereon, allowing a coating composition containing a paper powder and a material having an adhesive property to be deposited on at least a portion of a surface of the core portion to form a coated granulated matter, and drying the coated granulated material to produce a dried granulated matter having a water content equal to or lower than 12% by weight. Further, the present invention resides in a process for producing a granular excrement treating article, characterized by the steps of mixing, into a powder of an organic waste having a particle size equal to or smaller than 5 mm, an amount of a material smaller than that of the powder of the organic waste and having an adhesive property, granulating the resulting mixture to form a granulated matter having a grain size equal to or larger than 1 mm, allowing a copper compound or a zinc compound or a copper compound and a zinc compound each having a deodorizing function to be deposited on at least a portion of a surface of the formed granulated matter, thereby forming, as a core portion, a granulated matter having the copper compound or the zinc compound or the copper compound and the zinc compound deposited thereon, allowing a coating composition containing a paper powder and a material having an adhesive property to be deposited on at least a portion of a surface of the core portion to form a coated granulated matter, and drying the coated granulated matter to produce a dried granulated matter having a water content equal to or lower than 12% by weight. Yet further, the present invention resides in a process for producing a granular excrement treating article, characterized by the steps of mixing, into a powder of an organic waste having a particle size equal to or smaller than 5 mm, an amount of a material smaller than that of the powder of the organic waste and having an adhesive property, granulating the resulting mixture to form a granulated matter having a grain size equal to or larger than 1 mm as a core portion, allowing a coating composition containing a paper powder and a material having an adhesive property to be deposited on at least a portion of a surface of the core portion to form a granulated matter coated with the coating composition, allowing a copper compound or a zinc compound or a copper compound and a zinc compound each having a deodorizing function to be deposited on at least a portion of a surface of the coated granulated matter to form a coated granulated matter having the copper compound or the zinc compound or the copper compound and the zinc compound deposited thereon, and drying the coated granulated matter having the copper compound or the zinc compound or the copper compound and the zinc compound deposited thereon to produce a dried granulated matter having a water content equal to or lower than 12% by weight. Yet further, the present invention resides in a process for producing a granular excrement treating article, characterized by the steps of mixing, into a powder of an organic waste having a particle size equal to or smaller than 5 mm, an amount of a material having an adhesive property and an amount of an additive smaller than that of the powder of the organic waste, granulating the resulting mixture to form a granulated matter having a grain size equal to or larger than 1 mm as a core portion, allowing a coating composition containing a paper powder and a material having an adhesive property to be deposited on at least a portion of a surface of the core portion to form a granulated matter coated with the coating composition, allowing a copper compound or a zinc compound or a copper compound and a zinc compound each having a deodorizing function to be deposited on at least a portion of a surface of the coated granulated matter to form a coated granulated matter having the copper compound or the zinc compound or the copper compound and the zinc compound deposited thereon, and drying the coated granulated matter having the copper compound or the zinc compound or the copper compound and the zinc compound deposited thereon to produce a dried granulated matter having a water content equal to or lower than 12% by weight. Further, the present invention resides in a process for producing a granular excrement treating article, characterized by the steps of mixing, into a powder of an organic waste having a particle size equal to or smaller than 5 mm, an amount of a material having an adhesive property smaller than that of the powder of the organic waste, granulating the resulting mixture to form a granulated matter having a grain size equal to or larger than 1 mm as a core portion, allowing a coating composition containing a paper powder retaining a copper compound or a zinc compound or a copper compound and a zinc compound each having a deodorizing function and a material having an adhesive property to be deposited on at least a portion of a surface of the core portion to form a coated granulated matter as a granular core portion having a surface at least partially coated with the coating composition, and drying the coated granulated matter thus formed to produce a dried coated granulated matter having a water content equal to or lower than 12% by weight. Further, the present invention resides in a process for producing a granular excrement treating article, characterized by the steps of mixing, into a powder of an organic waste having a particle size equal to or smaller than 5 mm, an amount of a material having an adhesive property smaller than that of the powder of the organic waste, granulating the resulting mixture to form a granulated matter having a grain size equal to or larger than 1 mm as a core portion, allowing a coating composition containing a paper powder retaining a copper compound or a zinc compound or a copper compound and a zinc compound each having a deodorizing function and an additive to be deposited on at least a portion of a surface of the core portion to form a granulated matter as a granular core portion having a surface at least partially coated with the coating composition, and drying the coated granulated matter thus formed to produce a dried coated granulated matter having a water content equal to or lower than 12% by weight. Further, the present invention resides in a process for producing a granular excrement treating article, characterized by the steps of mixing, into a powder of an organic waste having a particle size equal to or smaller than 5 mm, an amount of a material having an adhesive property and an amount of an additive smaller than that of the powder of the organic waste, granulating the resulting mixture to form a granulated matter having a grain size equal to or larger than 1 mm as a core portion, allowing a coating composition containing a paper powder retaining a copper compound or a zinc compound or a copper compound and a zinc compound each having a deodorizing function and a material having an adhesive property to be deposited on at least a portion of a surface of the core portion to form a granulated matter as a granular core portion having a surface at least partially coated with the coating composition, and drying the coated granulated matter thus formed to produce a dried coated granulated matter having a water content equal to or lower than 12% by weight. Yet further, the present invention resides in a process for producing a granular excrement treating article, characterized by the steps of mixing, into a powder of an organic waste having a particle size equal to or smaller than 5 mm, an amount of a material having an adhesive property and an amount of an additive smaller than that of the powder of the organic waste, granulating the resulting mixture to form a granulated matter having a grain size equal to or larger than 1 mm as a core portion, allowing a coating composition containing a paper powder retaining a copper compound or a zinc compound or a copper compound and a zinc compound each having a deodorizing function, a material having an adhesive property and an additive to be deposited on at least a portion of a surface of the core portion to form a granulated matter as a granular core portion having a surface at least partially coated with the coating composition, and drying the coated granulated matter thus formed to produce a dried coated granulated matter having a water content equal to or lower than 12% by weight.

### BRIEF DESCRIPTION OF THE DRAWINGS

FIG. 1 is a schematic step diagram showing steps for producing a material having a deodorizing function according to an embodiment of the present invention. FIG. 2 is a schematic step diagram showing steps for producing a material having a deodorizing function according to an embodiment of the present invention different from FIG. 1. FIG. 3 is a schematic step diagram showing steps for producing a granular excrement treating article according to an embodiment of the present invention.

### BEST MODE FOR CARRYING OUT THE INVENTION

In the present invention, a granular excrement treating article is used for a human or an animal and formed into a coated granulated material comprising a granular core portion and a coating layer portion formed to cover the granular core portion. In the present invention, the granular core portion is formed into a granular material having a grain size equal to or larger than 1 mm to contain a powder of an organic waste, an amount of a material smaller than that of the power of the organic waste and having an adhesive property, a silver compound, a copper compound or a zinc compound or a metallic compound of two or more of these metals having a deodorizing function (which will be referred to as a metallic compound having a deodorizing function hereinafter). The coating layer portion is formed of a mixture a paper powder and a material having an adhesive property to cover the grains of the granulated matter as the granular core portion. In the granular excrement treating article according to the present invention, it is preferable that the surface of the coating layer portion is not coated with the metal compound having the deodorizing function, so that when a person or an animal touches the granular excrement treating article, it does not come into direct contact with the metal compound having the deodorizing function. In the present invention, it is possible to allow the metal compound having the deodorizing function to be deposited and retained on at least a portion between the granular core portion and the coating layer portion or a portion of the inside of the coating layer portion, or on a portion between the granular core portion and the coating layer portion and a portion of the inside of the coating layer portion. In the present invention, examples of the metallic compound having the deodorizing function include silver nitrate, silver phosphate, silver sulfate, copper chloride, copper nitrate, copper phosphate, copper sulfate, zinc nitrate, zinc chloride or zinc sulfate, a water-soluble compound and basic compound of any one of these metals, or silver hydroxide, copper hydroxide, zinc hydroxide or zinc oxide, or a mixture of two or more of these metallic compounds.

In the present invention, a water-soluble compound of copper or zinc or copper and zinc having a deodorizing function, or a water-insoluble complex, water-insoluble basic compound or water-insoluble hydroxide of copper or zinc or copper and zinc retained in the paper powder has a deodorizing effect, particularly, for hydrogen sulfide and ammonia, and has a function of removing obnoxious odor components by the chemical reaction. A copper and/or zinc compound having a deodorizing function is contained in or mixed into the granular core portion or the coating layer portion or the granular core portion and the coating layer portion of the granular excrement treating article.

In the present invention, among such copper and/or zinc compounds having the deodorizing function, a water-soluble copper compound includes a water-soluble copper compound, for example, such as copper chloride, a copper sulfate or copper nitrate, and a water-soluble zinc compound includes a water-soluble zinc compound, for example, such as zinc chloride, a zinc nitrate or zinc sulfate. Other copper or zinc compounds having a deodorizing function include basic compounds or hydroxides of copper and/or zinc. The basic compounds or hydroxides of copper and/or zinc can be produced by adjusting the pH of an aqueous solution of the water-soluble copper and/or zinc compound. In the present invention, if a solution, for example, of 12 grams of CuSO₄.5H₂O dissolved in 10 liter of water is used as a solution of copper sulfate contained in the granular core portion, the amount of copper sulfate contained in 1 ml of the solution of copper sulfate is 1.2 mg, and the copper content determined in terms of copper from this value is 0.3 mg. In the present invention, the amount of copper carried per 100 grams of the granular core portion is equal to or larger than 10 mg, preferably, equal to or larger than 50 mg, further preferably equal to or larger than 100 mg. It is preferable that the concentration and amount of the solution of copper sulfate sprayed to the granular core portion is determined by determining the content of water in grains of the coated granulated matter having the solution sprayed thereto by a test or the like. In the present invention, as regards silver and zinc, the amount can be determined according to a ratio of atomic weight to copper. The more the amount of the copper and/or zinc compound having the deodorizing function, the larger the deodorizing effect, which is desirable, but the copper and/or zinc compound having the deodorizing function is relative expensive, and if the amount of this compound used is increased, the cost is increased. Therefore, it is preferable that the amount of copper carried per 100 grams of the granular core portion is equal to or smaller than 300 mg, preferably equal to or smaller than 200 mg. In either case, if the amount of the metallic compound having the deodorizing function is smaller than a lower limit amount, intended deodorizing and sterilizing effects cannot be obtained and hence, the amount smaller than the lower limit amount is not preferred.

According to the present invention, copper or zinc ion can be deposited on carboxymethyl cellulose, and the carboxymethyl cellulose having the copper or zinc ion deposited thereon can be retained in or secure to a paper powder to provide a material having a deodorizing function. In the present invention, the paper powder is a powder having a particle size, for example, equal to or smaller than 0.35 mm, and includes, for example, a paper powder generated upon the cutting of a paper, a cutting wastage generated upon the production of a thin paper sheet, a reject article or cutting wastage of a thin paper sheet, a cutting wastage or a pulverized material of a classification product of a reject article generated upon the production of sanitary goods such as a paper diaper, or a pulverized material of a cutting wastage generated at a bookbinding step and at a sanitary paper producing step. Such a paper powder includes a paper powder having a particle size equal to or smaller than 0.35 mm and recovered by a dust collector, particularly, a paper powder having a particle size equal to or smaller than 0.1 mm.

The paper powder used in the present invention includes a toilet paper waste such as a cutting wastage or a reject article generated upon the production of a toilet paper, a tissue paper waste such as a cutting wastage or a reject article generated upon the production of a tissue paper, a facial tissue waste such as a cutting wastage or a reject article generated upon the production of a facial tissue, a coarse paper waste such as a cutting wastage or a reject article generated upon the production of a coarse paper, a cellulose wadding waste such as a cutting wastage or a reject article generated upon the production of a cellulose wadding, a pulverized material of a thin paper waste of a paper towel waste such as a cutting wastage or a reject article generated upon the production of a paper towel, or a mixture of two or more of pulverized materials of the paper wastes.

Further, a classification product rich in pulp resulting from the classification of the pulverized material of the sanitary goods waste may be used as the paper powder in the present invention. The classification product rich in pulp includes a classification product of a sanitary goods waste having a larger water absorbing capacity and rich in pulp, which is produced pulverizing sanitary goods waste such as a paper diaper waste, a breast pad waste, a menstrual napkin waste, a urine-absorbing pad waste or a combination of two or more of these sanitary goods wastes into a particle size equal to or smaller than 5 mm, classifying the resulting pulverized material of the sanitary goods waste to separate off a classification fraction rich in plastic, further classifying a classification product which is free of the classification fraction rich in plastic and which mainly contains a pulp and a water-absorptive resin to separate off the classification fraction rich in water-absorptive resin.

What can be used as the paper powder in the present invention further include, for example, a buffed powder, a pulverized material of a corrugated board waste, a pulverized material of a newspaper waste, a pulverized material of a mechanical pulp waste, a pulverized material of a chemical pulp waste, a pulverized material of a semi-chemical pulp waste, a pulverized material of a wood pulp waste, a paper powder generated upon bookbinding, a paper powder generated upon the production of a non-woven fabric, and a paper powder generated in a paper-making process, which has a smaller water absorbing capacity.

In the present invention, a paper powder having a particle size equal to or smaller than 0.35 mm is introduced into a conveyor, and an aqueous solution of a water-soluble compound of copper or zinc or copper and zinc is mixed into the paper powder. If citric acid is present in the aqueous solution of the water-soluble compound of copper or zinc at this mixing step, the water-soluble compound of copper or zinc is converted into a water-soluble citric complex salt and hence, the precipitation of the copper or zinc compound from the aqueous solution of the copper or zinc compound can be prevented. The aqueous solution of the compound of copper or zinc or copper and zinc mixed into the paper powder or the aqueous solution of the citric complex of copper or zinc or copper and zinc acid is mixed with the paper powder while being moved under agitation by a screw in a closed transferring path. The water-soluble citric complex of copper or zinc or copper and zinc mixed while being moved under the agitation within the screw conveyor and impregnated into the paper powder is mixed with an aqueous solution of a pH adjuster for the water-insolubilizing purpose. In this case, the paper powder impregnated with the aqueous solution of the copper or zinc or copper and zinc compound converted into the citric complex salt within the screw conveyor is discharged from an outlet of the screw conveyor into a tank or a screw conveyor having an aqueous solution of the pH adjuster in sodium hydroxide contained therein.

The aqueous solution containing the citrate of the water-soluble compound of copper or zinc or copper and zinc contained in the paper powder discharged from the screw conveyor is neutralized to near a pH value of 7 by an aqueous solution of sodium hydroxide and deposited to the paper powder in the form of a basic compound or hydroxide adhered to the paper powder. In this manner, the compound of copper or zinc or copper and zinc deposited to the paper powder is converted into the basic compound or hydroxide, thereby forming the paper powder having the compound of copper or zinc or copper and zinc deposited thereto. Then, the paper powder containing the hydroxide of copper or zinc or copper and zinc is transferred to a washing step, where it is washed and removed. The removed paper powder containing the hydroxide of copper or zinc or copper and zinc is dehydrated, pulverized, dried and graded to provide a product. In the present invention, the compound of copper or zinc or copper and zinc retained in the paper powder is a water-soluble inorganic acid salt, a water-insoluble basic salt, a hydroxide or an oxide, and depending on the application of the material having the deodorizing function, this compound can remain water-soluble without being converted into a water-soluble compound, in order to provide a product.

In the present invention, the content of copper ion or zinc ion or copper ion and zinc ion in the material having the deodorizing function is equal to or smaller than 10% by weight, preferably, in a range of 5 to 3% by weight, but can be equal to or smaller than 3% depending on the application of sterilization or deodorization. To effectively achieve the sterilizing or deodorizing effect, the content of copper ion or zinc ion or copper ion and zinc ion in the material having the deodorizing function is equal to or larger than 0.2% by weight. If the color of, for example, the copper ion is desired to be hidden using a dye or a pigment, then the content of copper ion or zinc ion or copper ion and zinc ion in the material having the deodorizing function is in a range of 0.5 to 2% by weight, preferably, in a range of 1 to 1.5% by weight in order to enhance the chroma of the dye.

The water-soluble or water-insoluble compound of copper or zinc or copper and zinc is used in a state in which it has been dissolved or suspended in water. However, the amount of water absorbed per unit area in the paper powder varies depending on the type of the paper powder and hence, it is preferable that a water absorbing capacity test is previously carried out for a paper powder used as a starting material, whereby a water absorbing capacity of the paper powder is measured, and an amount of the water-soluble compound of copper or zinc or copper and zinc impregnated is previously determined in accordance with an amount of aqueous solution absorbed by the paper powder. In the present invention, when the paper powder has a larger water absorbing capacity, the aqueous solution of compound of copper or zinc or copper and zinc used is impregnated in a large amount into the paper powder, and an amount of the compound of copper or zinc or copper and zinc corresponding to the impregnated amount is retained in the paper powder and possibly, may exceed a preset amount and hence, the concentration of the compound of copper or zinc or copper and zinc in the aqueous solution can be decreased. In the case of the paper powder having the larger water absorbing capacity as described above, a paper powder having a smaller water absorbing capacity can be mixed with such paper powder to adjust the amount of water absorbed per unit amount by the paper power to a smaller value, and the amount of aqueous solution used can be set at a suitable value. When the amount of water absorbed by the paper powder is smaller, the amount of aqueous solution impregnated is relatively small and hence, the concentration of the compound of copper or zinc or copper and zinc in the aqueous solution can be increased.

The paper powder having a larger water absorbing capacity includes powders of sanitary thin paper wastes such as a reject article and an end wastepaper produced upon the production of a sanitary thin paper such as a paper towel, a napkin paper, a facial tissue, a toilet paper, a coarse paper or cellulose wadding. The paper powder having a smaller water absorbing capacity includes powders of a printing paper or a packing paper containing a sizing agent mixed thereinto, so that water is difficult to permeate into the paper. If the water absorbing capacity is regulated to adjust the amount of compound of copper or zinc or copper and zinc retained in the paper powder, the amount of an aqueous solution of the compound of copper or zinc or copper and zinc used can be decreased to ensure a decrease in amount of water waste.

In the present invention, in order to convert the water-soluble compound of copper or zinc or copper and zinc into the water-insoluble compound of copper or zinc or copper and zinc, the pH value of the aqueous solution can be adjusted in a pH adjusting tank to a range of 6 to 7.5, preferably a range of 6.8 to 7.2 by a pH adjustor to convert the water-soluble compound of copper or zinc or copper and zinc into a water-insoluble basic compound or hydroxide of copper or zinc or copper and zinc. In the present invention, in order to neutralize the water-soluble compound of copper or zinc or copper and zinc by a smaller amount of a solution of a pH adjustor, the pH adjustor is used in the form of a solution having a relatively high concentration. In this manner, the amount of a liquid waste can be decreased by decreasing the amount of the pH adjuster solution. In the present invention, water-soluble alkali metal hydroxide, alkali metal carbonate, alkali metal bicarbonate or alkali earth metal hydroxide can be used as the pH adjustor, but in order to use any of these compounds in the form of a solution in a smaller amount to avoid the generation of carbon dioxide gas, it is preferable to use the water-soluble alkali metal hydroxide or alkali earth metal hydroxide.

In the present invention, it is preferable that the pH adjusting tank is formed as a reaction tank including a liquid reservoir of a possibly small volume. When the pH adjusting tank is of a screw conveyor type having a closed transfer pipe, the closed transfer pipe can be inclined to reduce the volume of the liquid reservoir within the pH adjusting tank, so that the amount of wastewater discharged from the pH adjusting tank can be decreased. The amount of the pH adjustor solution in the pH adjusting tank can be more decreased, as the closed transfer pipe is inclined at a smaller angle and a liquid overflow port is provided at a lower location. In the present invention, the water-soluble compound of copper or zinc or copper and zinc retained in the paper powder is neutralized in the pH adjusting tank and thus converted into a water-insoluble basic compound or hydroxide of copper or zinc or copper and zinc as remaining retained in the paper powder, and this water-insoluble basic compound or hydroxide is retained as it is. The paper powder having the water-insoluble basic compound or hydroxide of copper or zinc or copper and zinc retained therein is scraped upwards by the screw in the inclined closed transfer pipe, whereby the pH adjustor solution deposited on the paper powder is separated. The paper powder having the water-insoluble hydroxide of copper or zinc or copper and zinc retained therein and free of the pH adjustor solution separated is placed onto a belt conveyor and fed to a dehydrating zone, where the pH adjustor solution deposited is squeezed off by a squeeze roller. The paper powder having the water-insoluble hydroxide of copper or zinc or copper and zinc retained therein and free of the pH adjustor solution separated is pulverized in a pulverizing zone.

When the water-soluble compound of copper or zinc or copper and zinc is to be retained in the paper powder, the aqueous solution of the water-soluble compound of copper or zinc or copper and zinc can be mixed by sprinkling or spraying into the paper powder transported on a careen conveyor. The paper powder having the water-soluble hydroxide of copper or zinc or copper and zinc retained therein is squeezed by a squeeze roller provided at an end of the screen conveyor, whereby a surplus amount of deposited water is separated off from the paper powder. The paper powder free of the surplus water separated off, i.e., the wet paper powder having the aqueous solution of the compound of copper or zinc or copper and zinc retained therein is fed to a pH adjusting zone by a screen conveyor, where it is subjected to the neutralization by sprinkling or spraying of a pH adjusting solution. This causes the water-soluble compound of copper or zinc or copper and zinc retained in the paper powder to be converted into a water-insoluble hydroxide as remaining retained in the paper powder.

The paper powder having the water-insoluble hydroxide of copper or zinc or copper and zinc retained therein is squeezed by a squeeze roller, whereby a surplus amount of deposited water is separated off. The paper powder free of the surplus water separated off, i.e., the wet paper powder having the water-insoluble hydroxide of copper or zinc or copper and zinc retained therein is fed to a pulverized zone by a conveyor, where it is pulverized. In this process, screen openings of the screen conveyor have a size sufficient to ensure that particles of the paper powder are not leaked, and for example, a fabric having a particle-catching pore size equal to or smaller than 1μ can be used.

In the paper powder having the water-insoluble hydroxide of copper or zinc or copper and zinc retained therein, the water-insoluble hydroxide of copper has a blue color, and in order to hide this intrinsic color, the paper powder is colored into a proper color. When the paper powder having the water-insoluble hydroxide of copper or zinc or copper and zinc retained therein is to be colored, a coloring substance is used. The amount of coloring substance used is such that the coloring of the paper powder is not uneven. For example, when a deep coloring is desired, the amount of coloring substance used may be equal to or lower than 10% by weight, preferably, equal to or lower than 5% by weight, further preferably, equal to or lower than 3% by weight, based on the paper powder. In the case of a color such as red, brown green and blue colors, a relatively small amount of a coloring substance suffices. However, it is preferable that a coloring substance is used in an amount as large as the color of, for example, copper sulfate or copper hydroxide is hidden, because the coloring substance is expensive as compared with other starting materials.

In the present invention, if a dye is used as a coloring substance, then it can be mixed into a paper powder along with a water-soluble compound of copper or zinc or copper and zinc. On the other hand, if a pigment is used as a coloring substance, then it is deposited on surfaces of particles of a material having a deodorizing function after retaining of water-soluble hydroxide of copper or zinc or copper and zinc in a paper powder. In order to uniformly color the paper powder having the hydroxide of copper or zinc or copper and zinc retained therein by a pigment or dye, it is preferable that an amount of coloring substance incorporated into a paper powder is previously determined by previously coloring the paper powder by a previous coloring test or the like.

In the present invention, a dye or a pigment can be used as a coloring substance. In the colored paper powder, the color peculiar to the hydroxide of copper or zinc or the hydroxide of copper and zinc retained in the paper powder can be hidden, which can be dependent on a harmony with the furniture in a room, a sanitary feeling, a user's preference, a tint atmosphere and the like, and a material having a deodorizing function can be multicolored.

According to the present invention, a material having a deodorizing function is produced by containing a paper powder which contains a compound of copper or zinc or copper and zinc retained therein and which has a particle size equal to or smaller than 0.35 mm. Therefore, the material having the deodorizing function can be produced using an inexpensive waste as a retainer. Thus, it is possible to provide an inexpensive material having a deodorizing function, as compared with the prior art. In addition, according to the present invention, an aqueous solution of a compound of copper or zinc or copper and zinc is mixed into a paper powder, particularly, a paper powder having a water absorbing capacity adjusted to a suitable value, and the resulting mixture is mixed into an aqueous solution of a pH adjustor to form an insoluble basic compound or insoluble hydroxide of copper or zinc or copper and zinc. The formed insoluble basic compound or insoluble hydroxide is retained in the paper powder, and the resulting paper powder is dried and then pulverized into a particle size equal to or smaller than 0.35 mm to provide a material having a deodorizing function. Therefore, it is possible to produce the material having the deodorizing function by a relatively simple operation of impregnating the aqueous solution of the compound of copper or zinc or copper and zinc into the paper powder and converting the impregnated compound of copper or zinc or compound of copper and zinc into the hydroxide.

The material having the deodorizing function, which has been produced in the above manner, can be used, for example, as a deodorizing material for a granular excrement treating article for a human or an animal.

According to the present invention, the granular excrement treating article for a human or an animal is formed into a coated granulated material comprising a granular core portion and a coating layer portion formed to cover the granular core portion. Here, the granular core portion is formed into a granulated matter having a grain size equal to or larger than 1 mm and containing a powder of an organic waste and an amount of a material smaller than that of the powder of the organic waste and having an adhesive property. The coating layer portion is formed of a mixture of a paper powder and a material having an adhesive property to cover the granulated matter as the granular core portion. In the granular excrement treating article according to the present invention, a silver compound, a copper compound or a zinc compound or a metal compound of two or more of these metals, each having a deodorizing function (each of which will be referred to as a metal compound having a deodorizing function hereinafter), is deposited and retained in at least a portion between the granular core portion and the coating layer portion, in at least a portion of the coating layer portion or in at least a portion of a surface of the coating layer portion. Further, in the granular excrement treating article according to the present invention, the metal compound having the deodorizing function can be also deposited and retained in at least a portion between the granular core portion and the coating layer portion, in at least a portion of the coating layer portion or in at least a portion of a surface of the coating layer portion in addition to the above-described portion. Yet further, the metal compound having the deodorizing function can be also deposited and retained in at least a portion of the coating layer portion and in at least a portion of a surface of the coating layer portion. In the present invention, examples of the copper or zinc compound having the deodorizing function include a water-soluble compound or a basic metal compound such as copper chloride, copper nitrate, copper phosphate, copper sulfate, zinc nitrate, zinc chloride or zinc sulfate, or a water-insoluble compound such as copper hydroxide, zinc hydroxide or zinc oxide, or a mixture of two or more of the above-listed copper or zinc compounds.

In the present invention, the paper powder as a starting material for the coating composition for forming the coating layer portion includes, for example, a separation product containing a pulp and a water-absorptive resin and recovered from a waste of a sanitary material, particularly, a separation product having a water-absorptive resin content smaller than a pulp content, i.e., a pulp separation product can be used as a paper powder which is a starting material for a coating composition for forming the coating layer portion. In this case, the copper or zinc compound having the deodorizing function can be adhered or deposited directly to the coating layer portion containing the pulp separation product by sprinkling or spraying. In this way, according to the present invention, the copper compound or the zinc compound or the mixture of these two or more compounds can be sprayed in the form of an aqueous solution or suspension onto a surface of a granular matter forming a rolling core portion or a surface of a coating layer portion of a rolling coated granular matter, and an aqueous solution or suspension of the copper or zinc compound having the deodorizing function can be deposited on the surface, or permeated into the inside from the surface and retained in the inside. The metal compound having the deodorizing function and deposited and retained on the surface or the inside of the coated granular matter in this manner is dried and secured on the surface or in the inside of the coated granular matter. The metal compound can be applied uniformly at a suitable concentration to the entire surface of the coated granular matter by spraying an aqueous solution thereof prepared or diluted to a suitable concentration.

According to the present invention, the granular core portion may be formed of grains having a grain size equal to or larger than 1 mm, and the shape of the granular matter is not particularly limited. Therefore, the granular core portion may be, for example, of spherical grains, columnar grains, hollow grains, granulates, massive grains, powdery grains or other shaped grains. According to the present invention, the dried coated granular matter is a coated granular matter having a coating layer portion covering the granular core portion, and is formed on the granular core portion at a size larger than the granular core portion by a value corresponding to the coating layer portion, but it is preferable that the dried coated granular matter is formed as grains having a grain size equal to or larger than 2 mm, preferably equal to or larger than 3 mm.

According to the present invention, the granular matter or the granular core portion having the grain size equal to or larger than 1 mm can be produced by granulating a mixture to be granulated, in the presence of water. In the present invention, the mixture to be granulated can be produced by mixing a powder of an organic waste having a particle size equal to or smaller than 5 mm and an amount of a material smaller than that of the powder of the organic waste and having an adhesive property with each other. In the mixture to be granulated, the material having the adhesive property can be mixed so that the content is equal to or lower than 30% by weight, or equal to or lower than 10% by weight, or equal to or lower than 5% by weight. When the mixture to be granulated is used for granulation to form the granular matter, a water-absorptive resin can be mixed in the mixture to be granulated, so that the content thereof is equal to or lower than 30% by weight, or equal to or lower than 10% by weight, or equal to or lower than 5% by weight. When the mixture to be granulated is used for granulation to form the granular core portion of the coated granular matter, if the content of a water-absorptive resin in the granular core portion is larger, the granular shape of the granular core portion may be impaired by the swelling of the water-absorptive resin upon the absorption of water, resulting in a fear that the coated granular matter is ruptured. Therefore, it is preferable that the water-absorptive resin is mixed in the mixture to be granulated, so that the content thereof is equal to or lower than 30% by weight, or equal to or lower than 10% by weight, or equal to or lower than 5% by weight. The material having the adhesive property is used to bond particles of a powder of an organic waste as a starting material to one another or to particles of an additive to provide an intended water-absorbency and a stable shape to the granulated matter. It is preferable that the amount of material used is selected with the water absorbency, shape stability and the like of the granulated grains taken into consideration, and its range is previously determined experimentally or the like.

In the case of the coated granular matter, the content of the material having the adhesive property can be equal to or lower than 5% by weight based on the mixture to be granulated, and the granular core portion can be one having a weaker water-absorbency. In this case, the absorption of water is performed mainly in the coating layer portion, and the retention of water is performed mainly in the granular core portion. It is preferable that the material having the adhesive property and contained in the granular core portion is selected so that even if the granular core portion retains water, its shape is easily impaired. In the present invention, an amount of water required for the granulation is mixed into the mixture to be granulated, prior to the granulation. In the present invention, water is mixed into the mixture to be granulated, and the resulting mixture containing the water mixed is granulated by a granulating device. In the present invention, the granulating device used for the granulation includes, for example, a screw-type extrusion granulating device for granulating a mixture having a relatively high water content, and a roll-type extrusion granulating device and a ram-type extrusion granulating device such as a disk pelleter for granulating a mixture having a relatively low water content. However, the granulating device is not limited to these granulating devices, and another granulating device may be used. The other granulating device includes, for example, an extrusion granulating device of another type, a rolling granulating device of a dish type, a drum type or the like, and a compressing granulating device of a briquetting type or the like. In the present invention, when the granulation is carried out by the screw-type extrusion granulating device, the water content of the mixture to be granulated is in a range of 25 to 50% by weight, and when the granulation is carried out by the roll-type extrusion granulating device, the water content of the mixture to be granulated is in a range of 10 to 25% by weight.

In the present invention, the organic waste includes one or more plastic wastes, or one ore more organic wastes, or a mixture of one or more plastic wastes and one ore more organic wastes.

In the present invention, the powder of the organic waste is a powder of a plastic waste, or a powder of an organic waste, or a powder of a plastic waste and an organic waste. The powder of the plastic waste is produced by pulverizing one type or two or more types of plastic wastes into a particle size equal to or smaller than 5 mm. In the present invention, the plastic waste is preferred from viewpoints of the performance and cost, because the water retention characteristic of the plastic waste can be enhanced by pulverizing the plastic waste into the particle size equal to or smaller than 5 mm. In the present invention, the plastic waste includes, for example, a nonstandard reject article or cutting wastage generated upon the production of sanitary goods, and also includes a product generated upon the regenerating treatment of the reject article of the sanitary goods, or a product which is generated upon the regenerating treatment of the used sanitary goods and which is not utilized at a step of producing the sanitary goods and at a step of regenerating the sanitary goods. Therefore, in the present invention, the plastic waste is a discarded nonstandard reject article of plastic products manufactured, or a plastic waste such as an end waste, which is generated at a step of producing the plastic products and which cannot be utilized, as it is, as a starting material for the products, including a plastic waste discarded after the use of the plastic product.

In the present invention, one example of the plastic waste is a separation product rich in plastic waste, which is separated by classification and recovered from a waste containing a plastic waste, such as a breast pad waste, an incontinence pad waste, a paper diaper waste and a menstrual napkin waste. A material produced by pulverizing even such a separation product rich in plastic waste into a particle size equal to or smaller than 5 mm is lower in pulp content, but still has a water retention characteristic and can be used as a water-retainable material for the granular core portion of the granular excrement treating article. Another separation product separated by classification and recovered from the waste containing the plastic waste is a separation product rich in a water-absorptive resin, which has a good water-absorbency and can be used as a water-absorptive material. The separation product rich in a pulp and a water-absorptive resin can be used as an organic waste for the granular core portion and the coating layer portion of the granular excrement treating article.

The plastic waste used in the present invention refers to a powdery plastic waste rich in plastic, i.e., containing a plastic at a plastic content equal to or higher than 10% by weight, preferably, equal to or higher than 30% by weight. For example, such powdery plastic waste includes a pulverized material from a waste of an excrement treating article for an animal; a separation product rich in plastic separated by classification from a sheet waste for an animal; a pulverized material from a paper diaper waste; a separation product rich in plastic separated by classification from a paper diaper waste; a pulverized material from a menstrual napkin waste; a separation product rich in plastic separated by classification from a menstrual napkin waste; a pulverized material from an animal paper diaper waste; a separation product rich in plastic separated by classification from an animal paper diaper waste; a pulverized material from an animal menstrual napkin waste; a separation product rich in plastic separated by classification from an animal menstrual napkin waste; a pulverized material from a breast pas waste; a separation product rich in plastic separated by classification from a breast pas waste; a pulverized material from a breast pas waste; a separation product rich in plastic separated by classification from a breast pas waste; a pulverized material from a sweat pad waste; a separation product rich in plastic separated by classification from a sweat pad waste; a pulverized material from an incontinence pad waste; a separation product rich in plastic separated by classification from an incontinence pad waste; a pulverized material from an animal sheet waste; a separation product rich in plastic separated by classification from an animal sheet waste; a pulverized material from a mask waste; a pulverized material from a seat head cover waste; a pulverized material from a pillow cover waste; a pulverized material from a synthetic resin fiber waste; and a mixture containing two or more of the pulverized materials. If a separation product rich in plastic of the plastic wastes which is separated by classification is used, another separation product separated simultaneously by classification, i.e., a pulp and a water-absorptive resin can be used effectively as a fluff pulp and hence, the use of this separation product is preferred.

A further example of the plastic waste used in the present invention is a laminate paper waste. The laminate paper waste includes a reject or defective laminate paper discarded as a waste, a print refuse of a laminate paper, an end refuse of a laminate paper, a reject or defective laminate paper product or a laminate paper cuffing waste generated upon the production of a laminate paper product such a laminate paper tableware, or a mixture of two or more laminate paper wastes. In the present invention, however, the laminate paper waste also includes wastes of used disposable laminate paper tableware supplied to a meal. Further examples of the laminate paper waste used in the present invention are a reject article or an end refuse or a mixture thereof, of a non-woven fabric or a non-woven fabric processed-product made of a plastic fiber; or a reject article or an end refuse of a plastic film or a plastic film processed-product; or a reject article or an end refuse of a plastic film or a plastic sheet or a plastic sheet processed-product. These wastes include those containing a plastic such as polyethylene, polypropylene, rayon and synthetic rubber materials in the form of a film. In addition, because the plastic waste itself has a water absorbency, one of the plastic wastes, which contains a pulp or a paper powder of a material having a water absorbency, can be pulverized into a particle size equal to or smaller than 5 mm and mixed into a pulverized material of another plastic waste, and the resulting mixture can be used as a material for forming the core portion. In this case, such waste is used, pulverized into a particle size equal to or smaller than 5 mm, preferably, equal to or smaller than 3 mm, further preferably, equal to or smaller than 2 mm. If they are pulverized into such a particle size, the plastic film contained in this waste can used as a water-retaining material, because it is possible to easily retain water between the film particles to exhibit a water-retaining function.

In the present invention, it order to increase the water absorbency and the water-retaining property of the granular excrement treating article using an inexpensive material, it is preferable that a powdery organic waste resulting from the pulverization into a particle size equal to or smaller than 5 mm is mixed into a powdery plastic waste, and the resulting mixture is granulated. In the present invention, the powdery organic waste is a nonstandard reject article or cutting wastage generated upon the production of a product of an organic material, or a waste generated upon the regenerating treatment of such reject article and a waste generated upon the regenerating treatment of a used waste, which are not utilized in the process for producing the organic material product as well as which are not utilized in the process for regenerating the reject article and the waste. In the present invention, the pulverized organic waste is one resulting from the pulverization of one or more organic wastes into the particle size equal to or smaller than 5 mm. The organic waste used herein includes, for example, a separation product rich in pulp separated by classification or the like from a waste of a paper diaper waste for an animal, a separation product rich in pulp separated by classification or the like from a bedding sheet waste, a separation product rich in pulp separated by classification or the like from a mask waste, a separation product rich in pulp separated by classification or the like from a eye mask waste, a separation product rich in pulp separated by classification or the like from a seat head cover waste, a separation product rich in pulp separated by classification or the like from a pillow cover waste, a separation product rich in pulp separated by classification or the like from a paper diaper waste, a separation product rich in pulp separated by classification or the like from a menstrual napkin waste, a separation product rich in pulp separated by classification or the like from an animal menstrual napkin waste, a separation product rich in pulp separated by classification or the like from a breast pad, a separation product rich in pulp separated by classification or the like from a sweat pad waste, a separation product rich in pulp separated by classification or the like from a incontinence pad, a separation product rich in pulp separated by classification or the like from an animal sheet waste, a pulverized material of a laminate paper waste, a pulverized material of a cutting wastage of a laminate paper, a pulverized material of an end refuse of a laminate paper, a buff powder, a pulverized material of a corrugated board refuse, a pulverized material of newspaper refuse, a pulverized material of a magazine refuse, a pulverized material of a paper-making sludge, a pulverized material of a pulp sludge, a pulverized material of a non-woven fabric refuse, a pulverized material of a synthetic resin fiber waste, a pulverized material of a wood refuse, a pulverized material of wood shavings, a wood powder, a pulverized material of pieces resulting from the demolition of a building or house, a pulverized material of a waste from the new construction of a house, a paper powder, a pulverized material of a titanium paper waste, a pulverized material of an extraction residue of parched and milled coffee beans, a pulverized material of used tea leaves, a pulverized material of a vegetable refuse, kitchen garbage, a pulverized material of sludge such as activated sludge, a pulverized material of used tickets or a punching refuse, or a mixture of two or more of them. Any of them is used as being pulverized into a powdery material having a particle size, for example, equal to or smaller than 5 mm.

In the present invention, the vegetable refuse as the organic waste includes a leaf vegetable refuse, a stalk or stem vegetable refuse, a fruit vegetable refuse, flower vegetable refuse, a spicy vegetable refuse, a mushroom refuse, a wild plant refuse, a wild grass refuse, or a mixture of two or more of these refuses, and for example, includes (1) an unacceptable vegetable from a hydroponic farm factory, e.g., unacceptable vegetables of bean sprouts and vegetable artificially grown in shade from radish; (2) various kitchen remnants from vegetable fruit markets, e.g., skins of leaf vegetables such as white rape, lettuce, cabbage, spinach, leaf portions of root vegetables such as radish, carrot, burdock and turnip; (3) wasted portions of various vegetables such as skins of potato and skins of fruits from a feeding center and a diary dish factory; (4) discarded vegetables which are unacceptable products for various vegetables from a producing farmhouse; (5) discarded vegetables for adjustment of the cost for various vegetables in markets; (6) strained lees from vegetables for juices such as carrot and strained lees from fruits for juices such as apple and orange, or (7) cutting wastages or residues of various vegetables generated at a processing step for cut vegetables in a supermarket or the like, or a mixture of two or more of these refuses. In the present invention, the vegetable refuse further includes a distilling residue generated upon the production of a spirit such as sweet potato spirits, wheat spirits and rice spirits. In the present invention, the used tea leaves include residues resulting from the leaching or extraction of tea leaves by water or heated water of a high temperature, including used tea leaves in a dry state and water-containing used tea leaves in a wet state. The extraction residue of parched and milled coffee beans includes residues resulting from the leaching or extraction of parched and milled coffee beans by water or heated water of a high temperature, i.e., extraction residues, including extraction residues in a dry state and water-containing extraction residues in a wet state. In the present invention, if an extraction residue in a wet state is used as a starting material, then a portion of an amount of water required at the granulation can be furnished with an amount of water contained in the extraction residue, which is preferable.

Among the organic wastes, the organic waste having a larger water-absorbing ability includes a separation product rich in pulp separated by classification or the like from a waste of an excrement treating article for an animal, a separation product rich in pulp separated by classification or the like from a paper diaper waste, a separation product rich in pulp separated by classification or the like from a menstrual napkin waste, a separation product rich in pulp separated by classification or the like from a waste of a paper diaper for an animal, a separation product rich in pulp separated by classification or the like from a menstrual napkin for an animal, a separation product rich in pulp separated by classification or the like from a breast pad waste, a separation product rich in pulp separated by classification or the like from a sweat pad, a separation product rich in pulp separated by classification or the like from an incontinence pad waste, or a separation product rich in pulp separated by classification or the like from a waste of a sheet for animal, or a mixture of two or more of them. Such an organic waste contains a water-absorptive resin and hence, has a large water-absorbing ability, as compared with a waste containing only a pulp. When a mixture to be granulated is produced using an organic waste having a large water-absorbing ability as described above, the amount of organic waste incorporated in a granulated material can be increased to increase the water-absorbing ability.

On the other hand, among the organic wastes used for the organic waste-pulverized material, a vegetable refuse, a titanium paper waste, a used ticket, a punching refuse, a synthetic resin fiber waste, or pieces resulting from the demolition of a building or house, or a mixture of two or more of them, is relatively low in water-absorbing ability, but has a water-retention ability. Therefore, when any of such organic wastes is incorporated in a relatively small amount, the water-retention characteristic can be maintained without losing of the water absorbency. In the present invention, when the water-absorbing speed of a dried powder produced from a plastic waste is extremely low as compared with a pulp, a surfactant can be used in order to compensate for a point that the water-absorbing speed is poor. The surfactant is added into a material to be granulated, before the granulation. It is preferable that the amount of surfactant added is equal to or lower than 5% by weight, preferably, equal to or lower than 1% by weight of the material to be granulated. An anionic surfactant, a cationic surfactant, an amphoteric surfactant or a nonionic surfactant, all of which are commercially available, can be used as the surfactant. In this case, the surfactant may be used in the form of a diluted or undiluted solution produced by dissolving the surfactant in a solvent such as water. The dried granulated material is produced by granulating a mixture containing a solution of a surfactant added thereto to form a granulated material having a grain size equal to or larger than 1 mm and drying the granulated material. The adding of the surfactant ensures that the dried material produced by granulating of the pulverized material of the organic waste to can be easily wetted to water, and the speed of absorption of water in a dried granulated material produced from the pulverized material of the organic waste, i.e., the granular excrement treating article can be increased.

In the present invention, to enhance the water-absorbing ability and the water-retention ability of the granular excrement treating article, a water-absorptive resin, in addition to a water-containing water-absorptive resin, can be incorporated in the core portion. In the present invention, to ensure that grains wetted by urine or the like during the use of the granular excrement treating article are adhered to one another to form a massive material, it is preferable that a coating layer portion having such a nature that grains are wetted by urine or the like and adhered to one another, is formed to cover at least a portion of a surface of a granulated material. Such a coating layer portion can be formed from a material for forming a surface layer having such a nature that grains are wetted by urine or the like and adhered to one another. In the present invention, in order to form such a coating layer portion equivalent to the surface layer of the conventional granular excrement treating article, a paper powder and a material having an adhesive property, a water-absorptive resin, or a material having an adhesive property, or a mixture of a water-absorptive resin and a material having an adhesive property can be used as a coating composition.

When a granular excrement treating article is to be produced using a granular material having a large water absorbency, a highly water-absorptive resin and water may be mixed into a powder of a plastic waste or a powder of an organic waste, or a mixture of a powder of a plastic waste and a powder of an organic waste prior to the granulation to form a composition to be granulated, or a highly water-absorptive resin may be mixed into a powder of a plastic waste or a powder of an organic waste, or a mixture of a powder of a plastic waste and a powder of an organic waste, and water may be then mixed into the resulting mixture to form a mixture to be granulated, and this mixture may be passed through a granulating device and thus granulated. In the present invention, any water to be added to the mixture to be granulated can be used unless it contains a substance impeding the granulation and the water absorbency of the excrement treating article, a harmful substance, a substance emitting an offensive smell, or a remarkably coloring substance. Such water or an aqueous solution or suspension capable of being added to the mixture to be granulated include water containing a material having an adhesive property and dissolved therein, or water containing a silver compound, a copper compound or a zinc compound which is a metal compound having a deodorizing function, or these two or more metal compounds dissolved or suspended therein. In the present invention, the water-absorptive resin may be a so-called water-containing regenerated water-absorptive resin recovered in a non-dehydrated state after absorption of water when used, i.e., a recovered water-containing water-absorptive resin. The recovered water-containing water-absorptive resin contains a fairly large amount of water and hence, can be used in place of water.

When the recovered water-containing water-absorptive resin is used, it absorbs water at a high magnification, and when the recovered water-containing water-absorptive resin is in a liquid state or in a gelled state, it absorbs a large amount of water. Therefore, the mixing of a smaller amount of the water-absorptive resin and a large amount of water is achieved simultaneously, and the amounts of a new water-absorptive and water added can be decreased, which is preferred. When it is desired to provide the granular core portion with a large water absorbency, a highly water-absorptive resin is mixed into a powder of a plastic waste, or a powder of a plastic waste and a powder of an organic waste prior to the granulation, and water is mixed into the resulting mixture to form a mixture to be granulated, and this mixture is then granulated through a granulating device. When a mixture to be granulated is produced using a recovered water-containing water-absorptive resin as described above, the amount of water supplied to the mixture to be granulated from the outside can be decreased by a value corresponding to the amount of water contained in the water-containing water-absorptive resin. In this case, when a recovered water-containing water-absorptive resin containing water in an amount equal to or more than 30 times, preferably, 50 times its own weight is used, the recovered water-containing water-absorptive resin can be mixed into the mixture to be granulated, so that the amount of water contained in water-containing water-absorptive resin is equal to the amount of water contained in the mixture to be granulated. In the case of the mixture to be granulated containing the recovered water-containing water-absorptive resin mixed thereinto as described above, the recovered water-containing water-absorptive resin is dehydrated and regenerated with an activity left and hence, can be used as a water-absorptive resin in a granular excrement treating article, which is preferable. In the present invention, the water-containing water-absorptive resin absorbing water to assume a gelled state includes a water-containing water-absorptive resin or a water-containing highly water-absorptive resin, for example, recovered in one of two groups having different densities and sizes, into which a pulverized material of a used sanitary goods or another used product containing a water-absorptive resin, e.g., a starting material for a sanitary goods or another product containing a water-absorptive resin is separated by a separating means such as a wet classification. Such a regenerated water-absorptive resin contains a sterilizer such as sodium chlorate and sometimes, a component as contained in body fluids even in a very small amount. Such a regenerated water-absorptive resin functions as a lubricant, when a powder of a plastic powder having a particle size equal to or larger than 5 mm or a mixture of a powder of a plastic waste having a particle size equal to or larger than 5 mm and a powder of an organic waste having a particle size equal to or larger than 5 mm is subjected to an extruding granulation in a granulating device. Especially, a highly water-absorptive resin containing water to assume a gelled or liquid state can exhibit a lubricating effect, even if its amount is equal to or smaller than 1% by weight of the mixture to be granulated.

In the present invention, the recovered water-containing water-absorptive resin to be mixed prior to the granulation is one or two types of water-containing water-absorptive resins containing water in an amount equal to or larger than 50 times, preferably, equal to or larger than 300 times, further preferably, equal to or larger than 500 times its own weight. The water-absorptive resin containing a large amount of water absorbed therein assumes a gelled state or a viscous liquid state. Especially, the water-absorptive resin containing a large amount of water is in a gelled state nearer to a liquid state and hence, the supplying of the water-containing water-absorptive resin in the mixing of the water-absorptive resin can be conducted in a manner similar to the supplying of a liquid, which is preferable. In the present invention, when the water-containing water-absorptive resin is used for the adding of water and the water-absorptive resin to the mixture to be granulated, the amounts of water and the water-absorptive resin added to the mixture to be granulated can be decreased by values corresponding to the amounts of water and the water-absorptive resin added by use of the water-containing water-absorptive resin. The amount of water-absorptive resin incorporated in the mixture to be granulated for the granular core portion can be equal to or smaller than 1% by weight, preferably, equal to or smaller than 0.1% by weight, further preferably, equal to or smaller than 0.05% by weight based on the granulated material. In this manner, grains in a granular excrement treating article of a coated granulated material including a granular core portion formed of a granulated material containing a smaller amount of water-absorptive resin incorporated are at least wetted during use and adhered to one another through coating layers forming surface portions of the grains in the granular excrement treating article. However, the swelling of the granular core portion is small in the course of adhering of the grains to one another and hence, a stable agglomerate can be formed, which is preferable. In the present invention, in order to increase the water-retention ability and the water-absorbing ability of the granulated material per one grain, an organic waste containing a fiber such as a pulp can be mixed.

Even the highly water-absorptive resin containing water absorbed therein can be mixed into an organic waste such as a powdery plastic and function as a lubricant during the granulation, whereby the granulation can be achieved successfully. Moreover, the water-containing water-absorptive resin mixed into the mixture to be granulated is heated and dried along with the granular excrement treating article grains, whereby the water-absorbing performance is lost, but the water-containing water-absorptive resin can be present as a water-absorptive resin having a smaller swelling property. The water-containing water-absorptive resin is more easily mixed as it contains water in an amount enough to assume a liquid state. Therefore, when the water-absorptive resin containing water absorbed therein as described above is mixed into the mixture to be granulated prior to the granulation, the supplying of an amount of water required in the granulating course to the mixture to be granulated can be achieved in the course of mixing of the water-absorptive resin and hence, a step of mixing water into the mixture to be granulated by spraying or the like can be omitted, which is preferable. In the present invention, a reaction waste liquid and a washing waste liquid generated at a step of fixing a metal compound having a deodorizing function in a fiber such as a pulp can be used as an amount of water required for the granulation and mixed into the mixture to be granulated.

In the present invention, the coating composition contains one type or two or more types of paper powders and one type or two or more types of materials having an adhesive property, so that when the granular excrement treating article is wetted by an excrement such as urine, adjacent excrement treating grains can be bonded to each other. A powdery pulp material such as a nonstandard sanitary paper waste or a cutting wastage of a sanitary paper discharged from a sanitary paper producing process can be used as such a paper powder. Such powdery pulp material includes a nonstandard sanitary paper waste or a cutting wastage of a sanitary paper discharged from a sanitary paper producing process, for example, a thin paper, a thin paper waste, a sanitary paper, a sanitary paper waste, a toilet paper sheet, a toilet paper waste, a tissue paper sheet, a tissue paper waste, a facial tissue sheet, a facial tissue waste, a course paper sheet, a course paper waste, a cellulose wadding, a cellulose wadding waste, a paper towel, a paper towel waste, a toilet seat sheet waste, and a buff powder. Wastes other than them and capable of being used as the coating material include a newspaper, a newspaper refuse, a magazine refuse, a mechanical pulp, a mechanical pulp waste, a chemical pulp, a chemical pulp waste, a semi-chemical pulp, a semi-chemical pulp waste, a cotton-like pulp, a cotton-like pulp waste, a wood pulp, a wood pulp waste, a pulverized material of a used pulp, a paper powder, a fluff pulp, a water-absorptive fiber waste, a water-absorptive resin waste, a paper powder containing a water-absorptive resin, a paper powder generated upon the bookbinding, a paper powder generated upon the producing of a non-woven fabric, a paper powder generated at a paper-making process, a paper powder generated upon the production of a sanitary material, and a mixture of two or more of these pulverized materials. Any of these materials is used for the formation of the coating layer portion in a state in which it has been pulverized into a particle size equal to or smaller than 0.5 mm, preferably, equal to or smaller than 0.3 mm, further preferably, equal to or smaller than 0.1 mm.

In the present invention, the metal compound having the deodorizing function has a deodorizing effect, especially, against a hydrogen sulfide and ammonia, and is deposited or contained in the coating layer portion of the granular excrement treating article. In the present invention, the water-soluble copper compound having the deodorizing function and to be deposited in the coating layer portion includes a water-soluble copper compound, for example, such as copper chloride, copper sulfate and copper nitrate. Further, the water-soluble zinc compound which is the metal compound having the deodorizing function and to be deposited in the coating layer portion includes a water-soluble zinc compound such as zinc chloride, zinc nitrate and zinc sulfate. Another copper or zinc compound having the deodorizing function is, for example, a complex, basic compound or hydroxide of copper or zinc. The complex, basic compound or hydroxide of copper or zinc can be produced by the pH value of an aqueous solution of the water-soluble copper or zinc compound. In the present invention, for example, when a solution of 12 grams of 5-hydrated copper sulfate (CuSO₄.5H₂O) dissolved in 10 liters of water is used as a solution of copper sulfate to be deposited by spraying, the amount of copper sulfate contained in 1 ml of the solution of copper sulfate is 1.2 mg, and the amount of copper determined in terms of copper from this value is 0.3 mg. In the present invention, the amount of copper carried per 100 grams of the coating layer portion is equal to or larger than 10 mg, preferably, equal to or larger than 50 mg, further preferably, equal to or larger than 100 mg. The amount of copper carried per 100 square centimeters of the coating layer portion is equal to or larger than 1.6 mg, preferably, equal to or larger than 8 mg, further preferably, equal to or larger than 16 mg. It is preferable that the concentration and amount of the solution of copper sulfate sprayed onto the surface of the granular core portion and the surface of the coating layer portion are determined by determining the copper content and the water content of grains of the sprayed coating granular material by a test or the like. In the present invention, as regards silver and zinc, amounts depending on atomic weight ratios to copper can be used. The larger the amount of the metal compound having the deodorizing function, the larger than deodorizing effect is, which is preferable. However, the metal compound having the deodorizing function is relatively expensive, and as the amount of metal compound used is larger, the cost is more increased. Therefore, it is preferable that the amount of copper carried per 100 g of the coating layer portion is equal to or smaller than 300 mg, preferably, equal to or smaller than 200 mg. In either case, if the amount of the metal compound having the deodorizing function is smaller than a lower limit amount, it is not preferable, because the intended deodorizing and sterilizing effects cannot be obtained.

In the present invention, a substance having a deodorizing effect, i.e., a deodorizer can be mixed into the coating layer portion, thereby allowing the coating layer portion to retain the deodorizing effect, and ensuring that the amount of the metal compound having the deodorizing function to be deposited in the coating layer portion can be decreased. Examples of such deodorizer are zeolite, bentonite, catechins, chalcone, flavanone, flavone, flavonol, flavanonol, flavanol, isoflavone, anthocyans, Smelnark (a trade name, made by Tokai Kosan, Co.), Miyarabi (a trade name, sold by Kaikyu Kurabu), Pancil BA-210-1 (a persimmon extract) (made by Release Science Industries, Co.), Hiba oil, or a mixture of two or more of them. Examples of the deodorizer incorporated in the granular core portion are activated carbon, silica gel, wood vinegar, bamboo vinegar, an extraction residue of parched and milled coffee beans, used tea leaves, or a mixture of two or more of them. By incorporating any of these deodorizers into the granular core portion, it is possible to decrease the amount of a metal compound having a deodorizing function to be retained between the granular core portion and the coating layer portion, in at least a portion of the coating layer portion, or in at least a portion of a surface of the coating layer portion, or between the granular core portion and the coating layer portion as well as in at least a portion of the coating layer portion or in at least a portion of a surface of the coating layer portion, or in at least a portion of the coating layer portion and in at least a portion of a surface of the coating layer portion.

In the present invention, the granular excrement treating article has a color peculiar to its starting material, e.g., a light brown color or the like, but a coating composition containing a coloring substance having a color and incorporated therein can be deposited on the surface of the granular core portion, whereby a color such as a light brown color peculiar to a powder of an organic waste as a starting material can be hidden, and the surface of the granular excrement treating article can be colored into a proper color tone. If the granular excrement treating article is produced in this manner, it is possible to meet, for example, the harmony with furniture in a room, a sanitary feeling in a room, a user's preference and a tint atmosphere at the time of use of the granular excrement treating article, and it is also possible to multicolor the granular excrement treating article.

When the coating layer portion colored in this manner is formed, the amount of the substance having the color and the coloring effect, i.e., the coloring substance, is far smaller than that of the granular material and for this reason, the coloring is liable to be uneven. Therefore, in order to evenly color the granulated granular material, a paper powder previously colored by a coloring substance is incorporated into a coating material. It is preferable that the colored paper powder to be incorporated into the coating material comprises particles having a smaller particle size, because the color resulting from the coloring is more uniformized. For example, the paper powder as the coating material can be a powder having a particle size equal to or smaller than 0.35 mm (350 μm), but preferably, is a powder having a particle size equal to or smaller than 0.1 mm (100 μm), more preferably, a powder having a particle size equal to or smaller than 0.05 mm (50 μm). In the present invention, a dye or a pigment for coloring the coating layer portion are selected from those which are inert to the metal compound having the deodorizing function.

In the present invention, the granular material formed by the granulation contains water present at the granulation, and the coating composition supplied to the surface of the granular material by scattering or the like forms the coating layer portion on the surface of the granular material by virtue of such water. In the present invention, the colored paper powder contained in the coating composition is adhered to the granular material formed by the granulation to uniformly color the granular excrement treating article at a desired color tone. In the present invention, the granular material resulting from the granulation is coated with the coating composition containing the paper powder colored at a color tone different from that of the starting material and hence, it can be colored at a color tone different from that of the starting material.

In the present invention, the colored paper powder is a paper powder colored, for example, by a pigment or a dye or another colorant, and is mixed with a water-absorptive resin to form a coating composition. In the present invention, the coating layer portion formed by the coating composition forms an outer layer of a granular excrement treating article, and has an effect that when the granular excrement treating article is wetted by urine, grains in the granular excrement treating article are bonded to one another. In the present invention, for example, a substance having an adhesive property, i.e., an adhesive can be incorporated into the coating composition, so that the grains in the granular excrement treating article are bonded easily to one another through respective coating layers.

In the present invention, the adhesive forming the coating composition includes, for example, a paste such as a-starch, and a highly water-absorptive resin such as sodium polyacrylate. White potato starch, wheat starch, sweat potato starch, corn starch, tapioca starch, rice starch, dextrin, starch such as α-starches, acrylamide, polyvinyl alcohol, carboxymethyl cellulose, or sodium alginate can be used as such a paste functioning as an adhesive, and used alone or in combination of two or more of them. Other adhesives which can be used are polyvinyl alcohol, gelatin, a highly water-absorptive resin, vinyl ester, bentonite, polyvinyl alcohol (PVA) (Poval: a trade name), Pluran, casein and gelatin, which can be used alone or in the form of a mixture of two or more of them. Examples of alcohol-soluble adhesives are hydroxyethyl cellulose (HEC), hydroxypropyl cellulose (HPC), and polyvinyl pyrrolidone (PVP), which can be likewise used alone or in the form of a mixture of two or more of them. A gelatinizing agent such as gelatin and polyvinyl alcohol are capable of forming a firm granulated material from grains of a material to be granulated having a grain size equal to or smaller than 5 mm and hence, the use thereof is preferred.

In the present invention, when a granulated material formed by granulation is coated by a coating composition containing a colored paper powder to produce a colored granular excrement treating article, the granular excrement treating article can be formed so that a portion wetted by excretion after use of the granular excrement treating article for excretion is visible diaphanously and thus, an underlying color is visible in relief. In this case, because the underlying color of the coating layer portion is visible diaphanously in relief, the wetted portion can be distinguished from a dried portion in which an underlying color is not diaphanous. In this case, the color tone of the colored paper powder used for the coating layer portion can be differed from, or made lighter than the color tone of the granulated material formed by granulation, so that the color tone of the granulated material forming the core portion is visible diaphanously from the outside as a result of wetting.

In the present invention, when a powder of an organic waste as a starting material used for a granular excrement treating article has an offensive smell, the granulated material is dried so that the water content is equal to or lower than 12% by weight, preferably, equal to or lower than 10% by weight, further preferably, equal to or lower than 8% by weight, in order to moderate the offensive smell or the like of the organic waste and in order to render the granulated material sanitarily safe, and in order to provide a smooth feeling and further to prevent a mold and an uncomfortable smell from being generated. It is preferable that the dried coating granulated material is formed into a granular material having a grain size equal to or larger than 1 mm in order to prevent a dust from being generated. In addition, in order to prevent the offensive smell of the power of the organic waste from deteriorating the environment within a room and to ensure that the dried coating granular material is sanitarily safe, an additive, i.e., a substance having a sterilizing effect, a substance of a coloring effect, a substance having an adhesive effect or a combination of two or more of them can be mixed into the dried coating granular material.

In the present invention, the powdery plastic waste and the organic waste are pulverized into a particle size enabling the granulation to be easy in order to form a granular excrement treating article having a grain size equal to or larger than 1 mm, e.g., a particle size equal to or smaller than 5 mm, preferably, equal to or smaller than 3 mm. In the present invention, the pulverization can be carried out in any of a dry manner and a wet manner. When the powdery plastic waste is a separation product resulting from a wet classification or the like, it is in a wet state containing water and is pulverized as it is in a wet manner. When the organic waste has been left to stand and is in a state containing water, it can be pulverized in a wet manner. The pulverization of the waste in the wet manner is preferred, because no dust is generated. Therefore, even when the organic waste is a plastic waste having a smaller water-content, the pulverizing step can be carried out in a wet manner, and the mixing step can be also carried out in a water-containing state. The mixing of the powder of plastic waste or a mixture of the plastic waste and the organic waste with a water-containing water-absorptive resin can be carried out in the course of transportation, for example, by a screw feeder.

In the present invention, when the organic waste is the plastic waste, the content of the dried plastic waste in the dried granulated material is equal to or larger than 30% by weight, preferably, equal to or larger than 50% by weight. The dried organic waste mixed to the dried plastic waste is contained in an amount smaller than the dried powder of the plastic waste. The content of the dried powder of the organic waste is determined depending on the type of the organic waste. An organic waste having larger water absorbency and water retention characteristics can be contained in a relatively large amount. However, as regards an organic waste having a smaller water retention characteristic, the content of a powdery plastic waste can be increased, and the content of the organic waste can be decreased, for example, to equal to or lower than 30% by weight and further to equal to or lower than 10% by weight.

It is preferable that the granular excrement treating article according to the present invention comprises a granulated material having into a grain size, for example, equal to or larger than 1 mm produced by the granulation, in order to avoid the generation of a dust and the scattering due to the disintegration or the peeling of the granular excrement treating article. However, if grains having a grain size, for example, equal to or larger than 3 mm the granulation into are produced by the granulation, the grains are difficult to scatter into a room, for example, from a box for a toilet, and even if the grains are scattered, they are easy to pick up and gather, which is preferable for maintaining the sanitation within a room. In these cases, however, the presence of grains having a grain size equal to or smaller than 1 mm and the presence of grains having a grain size equal to or smaller than 3 mm are not eliminated completely. In the present invention, the granulated material and the coated granulated material can be formed into a spherical shape, a columnar shape, a particulate shape or another shape or a mixture of them.

In the present invention, the water-absorptive resin means a water-absorptive resin having a low water-absorbency in addition to the highly water-absorptive resin. Therefore, the water-absorptive resin is a resin which absorbs water in an amount of several ten times to two thousand times the weight of the resin by weight to become a gelled state while being maintained in shape, or into a liquid state. Such a water-absorptive resin includes commercially available highly water-absorptive resins. Such highly water-absorptive resins are, for example, starch-based highly water-absorptive resins such as a starch-acrylonitrile graft copolymer, a starch-acrylic acid graft copolymer, a starch-styrene sulfonic acid graft copolymer and a starch-acrylamide graft copolymer; polyvinyl alcohol-based highly water-absorptive resins such as a cellulose-acrylonitrile graft copolymer, a cellulose-styrene sulfonic acid graft copolymer and a cross-liked carboxymethyl cellulose-acrylamide graft copolymer; polysaccharide-based highly water-absorptive resins such as hyaluronic acid and agarose; protein-based highly water-absorptive resins such as collagen; polyvinyl alcohol-based highly water-absorptive resins such as a polyvinyl alcohol cross-linked polymer and a thawed elastomer of an iced water-absorbed gel of polyvinyl alcohol; acrylic highly water-absorptive resins such as a sodium acrylate cross-linked polymer and sodium acrylate-vinyl alcohol copolymer; polyether-based highly water-absorptive resins such as a polyethylene glycol-diacrylate cross-linked polymer; addition polymer-based highly water-absorptive resins such as maleic acid anhydride-based (co)polymer and a vinyl pyrrolidone-based (co)polymer; and highly water-absorptive condensation resins, as well as a saponified product of a vinyl ester and an ethylenic unsaturated carboxylic acid or its derivate; a partially hydrolyzed product of polyacrylonitrile; a cross-linked product of polyethylene glycol; a saponified product of a vinyl ester and an ethylenic unsaturated carboxylic acid or its derivate; and a salt of chitosan; or gel of Pluran. These highly water-absorptive resins can be incorporated along or in combination of two or more of them into a powdery plastic waste or a mixture of a powdery plastic waste and a pulverized material of an organic waste having a particle size equal to or smaller than 5 mm prior to the granulation.

In the excrement treating article according to the present invention, a substance having a sterilizing effect can be added to the granulated product of the dried pulverized material of the organic waste having a particle size equal to or smaller than 5 mm. In the present invention, it is preferable that such a substance having a sterilizing effect is one having no decoloring effect to prevent the dried powdery plastic waste or a mixture of the dried powdery plastic waste and the pulverized material of the organic waste from being decolored. It is preferable that such a substance having a sterilizing effect is, for example, a food preserving and sterilizing substance used for the preservation and the sterilization of a food. A sterilizer, an antiseptic agent and a fungicide such as an organic acid, table salt, sorbic acid or its salt, calcium propionate, hinokitiol and benzoic acid or its salt can be used as such a substance having a sterilizing effect, and an anti-fungus agent can be also used. In the present invention, other examples which can be used as the substance having the sterilizing effect are acetic acid, succinic acid, lactic acid, malic acid, citric acid, tartaric acid, adipic acid, benzoic acid, sodium benzoate, sorbic acid, potassium sorbate, dehydroacetic acid, sodium dehydroacetate, propionic acid, sodium propionate, calcium propionate, hinokitiol, sodium hypochlorite, isobutyl para-oxybenzoate, isopropyl para-oxybenzoate, ethyl para-oxybenzoate, butyl para-oxybenzoate, propyl para-oxybenzoate, chlorine dioxide, table salt, or a mixture of two or more of them.

When a pigment which is not easily decolored by oxidation is used as a coloring substance for the dried powdery plastic waste and the dried pulverized material of the organic waste having a particle size equal to or smaller than 5 mm, sodium hypochlorous acid or its salt, e.g., sodium hypochlorite or calcium hypochlorite or chlorine dioxide can be used. The addition of hinokitiol which is an effective constituent in while-cedar oil is carried out by adding a wood powder containing hinokitiol such as hinoki and white-cedar. The use of the wood powder containing hinokitiol is preferred, because a waste from a lumbering factory can be utilized effectively.

In the present invention, the coating composition for forming the coating layer portion is a mixture of a paper powder and a water-absorptive resin, but in some cases, an additive can be further mixed to prepare a coating composition. In the present invention, the coating composition may be produced from a colored paper powder, a paper powder and a water-absorptive resin. In this case, the amount of the paper powder for forming the coating layer portion is equal to or larger than 50 parts by weight, preferably, 60 & by weight, further preferably, 55% by weight, based on the coating layer portion. The amount of the water-absorptive resin for forming the coating layer portion is 40 % by weight, preferably, 35% by weight, based on the coating layer portion. If sodium acrylate is used as a water-absorptive resin for forming the coating layer portion, particles are firmly bonded to one another in a course of forming the coating layer portion. Therefore, it is preferable that the amount of sodium acrylate used is equal to or lower than 30% by weight, and the shortage of a bonding force is compensated for by substituting a starch-based highly water-absorptive resin for the water-absorptive resin. If the content of the water-absorptive resin in the coating layer portion of the granular excrement treating article is equal to or lower than 5% by weight, the wetted coating layers in the wetted granular excrement treating article are adhered and bounded to one another, but grains in the excrement treating article bonded are disintegrated in water, so that they can be unbounded into the original pieces. In the coating layer portion, an adhesive such as glue can be substituted for a portion of the water-absorptive resin. If the content of the water-absorptive resin in the coating layer portion is decreased and the amount of the adhesive such as glue is increased, the binding function of the granular excrement treating article when wetted can be strengthened, and the function of dispersion in water can be increased.

In the granular excrement treating article according to the present invention, the surfaces of the grains containing the plastic powder or the plastic powder and the powder of the organic waste can be formed to become coated with the coating layers. During use of the granular excrement treating article, for example, urine is adhered to the surfaces of the coating layers in the granular excrement treating article, and the grains in the granular excrement treating article wetted by the urine adhered to the coating layers are bounded to one another through the wetted coating layers. In the case of solid excrement, when the grains are adhered to the solid excrement, they are bounded to one another so as to wrap the excrement. In the present invention, because the grains in the granular excrement treating article are bounded to one another so as to wrap the excrement, the offensive smell or the like of the excrement is adsorbed to the granular excrement treating article and is not emanated to the surroundings. As described above, in the present invention, the glue such as polyvinyl alcohol and wheat flour is mixed as an adhesive in the coating layer portion of the granular excrement treating article and hence, the coating layers are adhered to a human's or animal's excrement to wrap the excrement in a massive shape, and thus, the post-treatment of the granular excrement treating article is simple and easy. If an auxiliary deodorizer and/or a highly water-absorptive resin are or is mixed in the core portion of the excrement treating article for an animal, then the excrement treating article exhibits a further excellent deodorizing property as well as further excellent water absorbency and water retention. In the present invention, any of various pulp materials or any of various water-absorptive resins can be incorporated in addition to the paper powder into the coating layer portion formed on the core portion, so that coating layer portion has moderate water-absorbing and adhesive functions. In this case, the coating layer portion can be formed by incorporating a water-absorptive resin having water-absorbing and adhesive functions, a highly water-absorptive resin having a relatively large amount of water absorbed and a water-absorptive resin functioning as a thickener, for example, in addition to a pulp material powder or a paper powder, in an appropriate ratio determined experimentally.

In the present invention, the particle size of the plastic powder and the powder of the organic waste which are starting materials for the granular excrement treating article is equal to or smaller than 5 mm, preferably, equal to or smaller than 3 mm. Especially, when the particle size of the starting material is equal to or smaller than 5 mm, it is preferred because the water retention of the dried granulated material is increased. When the particle size of the starting material is equal to or smaller than 3 mm, it is preferred because the water retention of the dried granulated material is further increased. In the present invention, the powder of the organic waste can be mixed in an amount equal to or lower than 40% by weight, preferably, in an amount equal to or lower than 10% by weight into the plastic powder prior to the granulation.

According to the present invention, a plastic powder and an organic waste powder which have been conventionally discards whose treatment has been a problem, are mixed with a water-containing water-absorptive resin, and the resulting mixture is granulated, thereby forming a granular excrement treating article. More specifically, for example, used tea leaves, activated carbon and another burnable deodorizer are mixed into a pulverized matter of a starting material having a particle size equal to or smaller than 5 mm, and the resulting mixture is granulated to form a granular material having a grain size equal to or larger than 1 mm, preferably, equal to or larger than 3 mm. The granular material is coated with a coating composition containing a pulp or a paper powder and a highly water-absorptive resin as well as colored inorganic material grains to form a uniformly colored coating layer portion on a surface the granular material, thus forming a granular excrement treating article for a human or an animal. The granular excrement treating article is formed mainly of burnable materials, absorbs urine very well, has a good water-retention and a remarkably good deodorizing property, and moreover, is easy to discard by burning or the like. The granular excrement treating article according to the present invention is formed mainly of burnable materials and moreover, contains mainly a pulp or a paper powder having a high calorific value and a plastic having a high calorific value and hence, it has a larger calorific value and is easy to incinerate after use thereof.

Examples of the mode for carrying out the present invention will now be described with reference to the accompanying drawings, but the present invention is not limited to the following description and illustrations in any way.

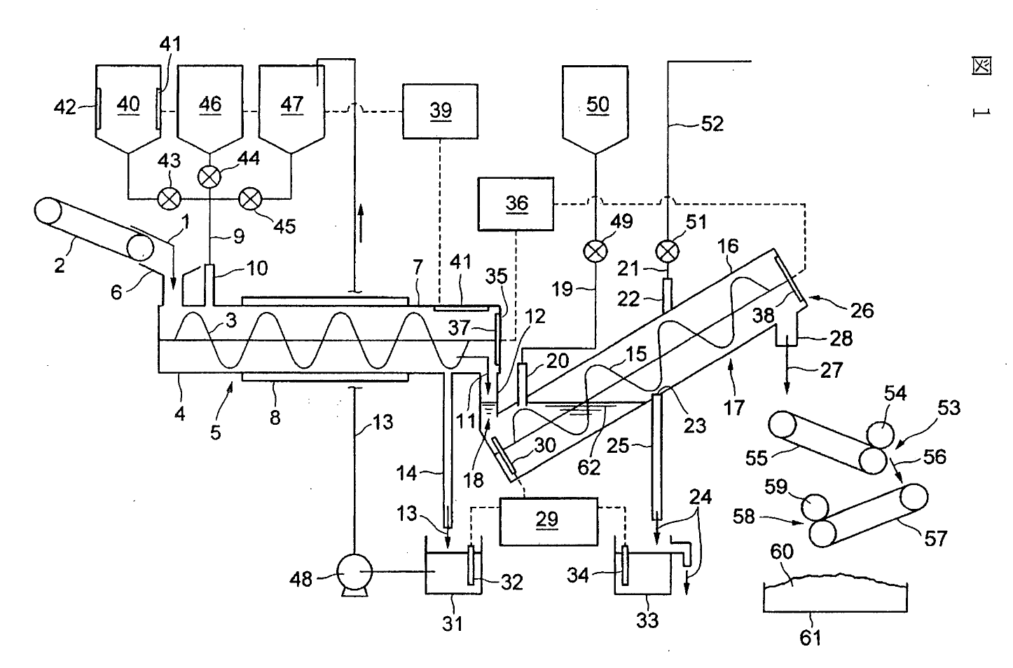

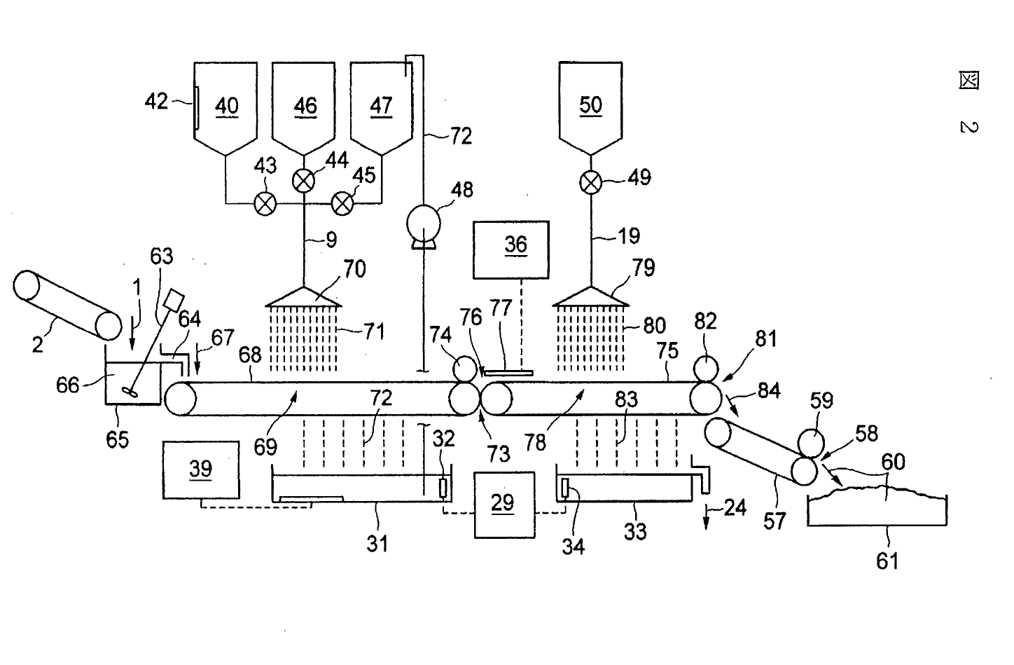

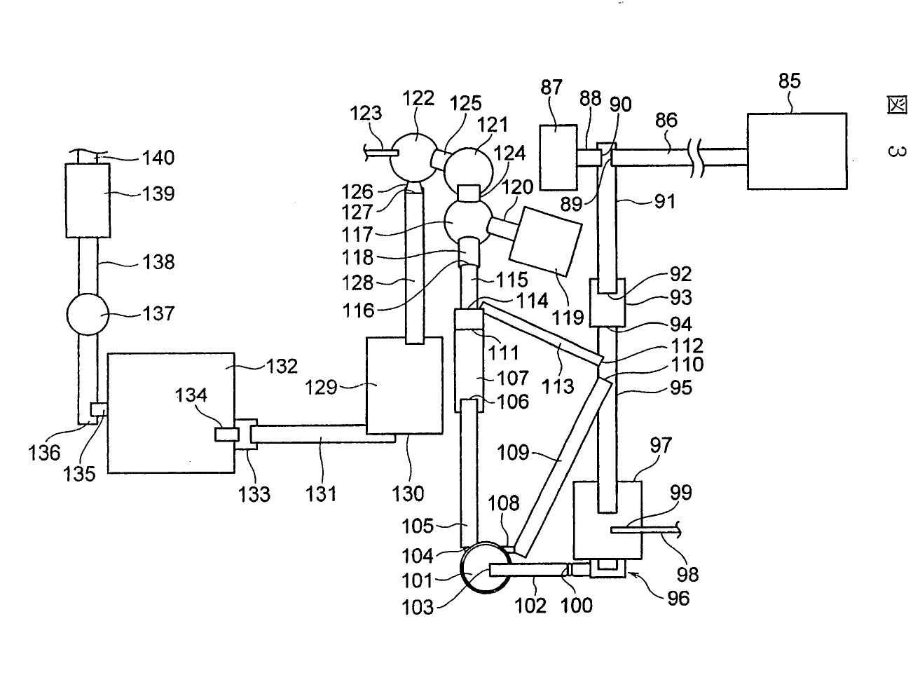

FIG. 1 is a schematic illustration showing a process for producing a material having a deodorizing function according to an embodiment of the present invention. FIG. 2 is a schematic illustration showing a process for producing a material having a deodorizing function according to an embodiment of the present invention different from FIG. 1. FIG. 3 is a schematic illustration showing a process for producing a granular excrement treating article according to an embodiment of the present invention. In FIGS. 1 and 2, like reference characters are affixed to corresponding portions or components.

Referring to FIG. 1, a belt conveyer 2 for supplying a paper powder 1 is connected to an upper portion of a paper supply hopper 6 of a screw mixing device 5 comprising a transporting pipe 4 having a screw agitating member 3 provided therein. The screw mixing device 5 includes a heating jacket 8 mounted to surround an outer wall 7 of the transporting pipe 4, and a compound solution introducing inlet 10 which is provided in vicinity of and downstream of the paper powder supply hopper 6, and to which an introducing pipe 9 for an aqueous solution of a copper or zinc or copper and zinc compound (which will be referred to as an aqueous solution of compound hereinafter) is connected. A discharge outlet 12 for a paper powder impregnated with the aqueous solution of compound is provided at a downstream end of the screw mixing device 5, and a compound solution discharge pipe 14 for discharging a surplus amount of the aqueous solution of compound is provided at a location upstream of the discharge port 12.

In this embodiment, the discharge port 12 for the paper powder 11 impregnated with the aqueous solution of compound is connected to an introducing portion 18 for the paper powder impregnated with the aqueous solution of compound in a screw neutralizing device 17 including a transporting pipe 16 having a screw agitating member 15 provided therein. An aqueous pH adjuster solution introducing pipe 20, to which an aqueous pH adjuster solution introducing pipe 19 is connected, is provided at a location downstream of the introducing portion 18 for the paper powder impregnated with the aqueous solution of compound in the screw neutralizing device 17. A washing water introducing pipe 22, to which a washing water supply pipe 21 is connected, is provided at a location downstream of the aqueous pH adjuster solution introducing pipe 20 of the screw neutralizing device 17, and a washing water waste discharge pipe 25 for discharging a washing water waste 24 is provided on a bottom upstream of the washing water introducing pipe 22 with its opening 23 protruding to above the bottom. At a downstream end 26 of the screw neutralizing device 17, there is a removal port 28 for a paper powder 27 containing the hydroxide of copper or zinc or copper and zinc compound retained therein (which will be referred to as a hydroxide-retaining paper powder hereinafter).

In the present embodiment, a pH measuring terminal 30 of a pH control device 29 is provided in the screw neutralizing device 17 at a location adjacent the introducing portion 18 for the paper powder impregnated with the aqueous solution of compound. In the present embodiment, the pH control device 29 further includes a pH measuring terminal 32 for measuring a pH value of a surplus aqueous solution of compound in a surplus compound solution recovery tank 31 to which the surplus compound solution discharge pipe 14 is connected, and a pH measuring terminal 34 for measuring a pH value of a washing water waste in a washing water waste storing tank 33 to which the washing water waste discharge pipe 25 is connected.

In the present embodiment, at a discharge end 35 of the screw mixing device 5 for the paper powder 11 impregnated with the aqueous solution of the compound and at a hydroxide-retaining paper powder removal end 26 of the screw neutralizing device 17, there are respectively water-content adjusters 37 and 38 of a water-content control device 36 for the discharged paper powder. In the present embodiment, a temperature control device 39 is mounted, which includes temperature adjusters 41 provided on a sidewall of a copper or zinc or copper and zinc compound dissolving and supplying tank 40 and on a ceiling of the discharge end of the screw mixing device 5 for the paper powder impregnated with the aqueous solution of compound, so that the temperatures within the screw mixing device 5 and the compound dissolving and supplying tank 40 are adjusted to, for example, 30 to 40°C., e.g., 35°C. In the present embodiment, a heating device 42 is mounted in the compound dissolving and supplying tank 40 in order to promptly conduct the dissolving of the compound in the compound dissolving and supplying tank 40. The compound dissolving and supplying tank 40, a dye dissolving and supplying tank 36 and a recovered-compound solution supply tank 47 are connected to the compound solution introducing pipe 10 through on-off valves 43, 44 and 45, respectively. In the present embodiment, a recovered surplus aqueous solution of compound 13 is fed from the surplus compound solution recovery tank 31 to the recovered-compound solution supply tank 47 by a feed pump 48.

In the present embodiment, the pH adjuster aqueous solution supply pipe 19 is connected to a pH adjuster aqueous solution supply tank 50 through an on-off valve 49. The pH control device 29 controls the opening and closing or the degree of opening of the on-off valve 49, based on pH values measured by the pH measuring terminals 30, 32 and 34 to control the pH value of an aqueous solution of a pH adjuster in the screw neutralizing device 17. The washing water supply pipe 22 is connected to a washing water supply source (not shown) through a washing water supply conduit 52 including an on-off valve 51.

A dehydrating belt conveyer 55 having a squeeze roller 54 at a discharge end 53 is connected to the removal port 28 for removing the hydroxide-retaining paper powder 27, so that the hydroxide-retaining paper powder 56 pressed and dehydrated by the squeeze roller 54 of the dehydrating belt conveyer 55 is discharged from the discharge end 53. A pulverizing belt conveyer 57 is mounted below the discharge end 53 of the dehydrating belt conveyer 55. The pulverizing belt conveyer 57 includes a pulverizing roller 59 at a pulverized matter discharge end 58, so that the hydroxide-retaining paper powder 56 dehydrated and discharged from the discharge end 53 of the dehydrating belt conveyer 55 is pulverized by the pulverizing roller, and the resulting pulverized matter 60 is stored as a semi-finished product before dried in a semi-finished product tank 61.

The present embodiment is arranged as described above and hence, a material having a deodorizing function can be produced using a paper waste made by pulverizing, for example, a reject thin paper discharged from a thin paper making process into a particle size equal to or smaller than 0.35 mm. A smaller particle size results in a larger specific surface area and hence, a smaller particle size, for example, as small as 0.1 mm or less is preferred.

In this case, the paper powder 1 is supplied from the paper powder supplying belt conveyer 2 through the paper powder supply hopper 6 to the screw mixing device 5. On the other hand, the copper or zinc or copper and zinc compound is introduced along with citric acid into the compound dissolving and supplying tank 40, where it is dissolved in water. A dye is also introduced into the dye dissolving and supplying tank 46, where it is dissolved in water. A surplus aqueous solution of compound recovered from the surplus compound solution recovery tank 31 is introduced into the recovered compound solution supply tank 47 by the feed pump 48. An aqueous solution of compound prepared by mixing together the aqueous solution of compound in the compound dissolving and supplying tank 40, the aqueous solution of the dye in the dye solution dissolving and supplying tank 46 and the recovered aqueous solution of compound in the recovered compound solution supply tank 47 by opening the on-off valves 43, 44 and 45 is supplied via the compound solution introducing pipe 9 and through the compound solution introducing port 10 to the paper powder supplied to the screw mixing device 5, where it is mixed into the paper powder.

The paper powder having the aqueous solution of compound mixed therein is transported in the transporting pipe 5, while being agitated by the screw agitating member, whereby it is impregnated with the aqueous solution of compound. The paper powder 11 impregnated with the aqueous solution of compound is discharged from the compound solution-impregnated paper powder discharge outlet 12 of the screw mixing device 5, and introduced into the compound solution-impregnated paper powder introducing portion 18 and then introduced into the aqueous solution 62 of pH adjuster present in the screw neutralizing device 17. The compound retained in the paper powder is neutralized by the aqueous solution of pH adjuster and thus converted into hydroxide as retained in the paper powder. The paper powder 27 retaining the hydroxide is removed from the hydroxide-retaining paper powder removal port 28 of the screw neutralizing device 17 and transferred onto the dehydrating belt conveyer 55, where it is dehydrated.

The dehydrated hydroxide-retaining paper powder 56 is transferred onto the pulverizing belt conveyer 57 where it is pulverized, and the pulverized matter 60 is stored as a semi-finished product in the semi-finished product tank 1. The dehydrated hydroxide-retaining paper powder is dried for commercialization, but if the hydroxide of copper or zinc or copper and zinc is firmly not retained in the paper powder, then the hydroxide of copper or zinc or copper and zinc can be retained in carboxymethyl cellulose (CMC), and the resulting matter can be retained in the paper powder. This enables the firm retaining of the hydroxide of copper or zinc or copper and zinc in the paper powder.

An embodiment shown in FIG. 2 is different from the embodiment shown in FIG. 1 in that in the embodiment shown in FIG. 1, the paper powder is mixed with the aqueous solution of compound of copper or zinc or copper and zinc and impregnated with the aqueous solution of compound using the screw conveyer system, but in the embodiment shown in FIG. 2, the paper powder is impregnated with the aqueous solution of compound by sprinkling or spraying the aqueous solution of compound of copper or zinc or copper and zinc to the paper powder using the screw conveyer system.

In the embodiment shown in FIG. 2, a belt conveyer 2 for supplying a paper powder 1 extends above a dispersing tank 65 including an agitating device 63 and an overflow outlet 64. The paper powder 1 is introduced from the paper powder supplying belt conveyer 2 into water already supplied into the dispersing tank 65, and is then agitated sufficiently to form a dispersion 66 of the paper powder. The dispersion 66 of the paper powder overflows in a direction of an arrow 67 from the overflow outlet 64 of the dispersing tank 65, and is then supplied onto a screen of a compound solution impregnation screen conveyer 68.

The dispersion 66 of the paper powder supplied onto the screen of a compound solution impregnation screen conveyer 68 is subjected to a filtering, whereby water therein is removed and as a result, a water-impregnated paper powder layer (not shown) is formed on the screen of the screen conveyer 68. The water-impregnated paper powder layer is moved along with the screen of the screen conveyer 68 to a compound solution-sprinkling zone 69, and an aqueous solution of compound 71 is sprinkled onto the water-impregnated paper powder layer from a compound solution sprinkling device 70 mounted above the zone 69, thereby forming a paper powder layer impregnated with the aqueous solution of compound.

A surplus amount of the aqueous solution of compound 72 is passed through the paper powder layer and the screen of the compound solution impregnation screen conveyer 68 and recovered into a surplus compound solution recovery tank 31. The recovered surplus aqueous solution of compound 72 is fed from the surplus compound aqueous solution recovery tank 31 into the recovered-compound solution supply tank 47 by the feed pump 48. On the other hand, the paper powder layer impregnated with the aqueous solution of compound and formed on the screen conveyer 68 and is moved along with the screen of the compound solution impregnation screen conveyer 68 is subjected to a pressing separation by a squeeze roller 74 mounted at the discharge end 73, whereby a surplus amount of the aqueous solution of compound is separated off. The compound solution-impregnated paper powder layer with the surplus amount of the aqueous solution of compound separated off is fed to a pH adjusting screen conveyer 75.

In the present embodiment, a water-content adjusting device 77 is mounted at an inlet 76 of the pH adjusting screen conveyer 75, so that the content of water in the compound solution-impregnated paper powder layer is adjusted to a predetermined value. The compound solution-impregnated paper powder layer having a water content adjusted is moved along with the pH adjusting screen conveyer 75 to a pH adjuster solution sprinkling zone 78, and an aqueous solution of a pH adjuster is sprinkled onto the compound solution-impregnated paper powder layer from a pH adjuster solution sprinkling device 79 mounted above the zone 78 to adjust the pH value of the compound solution-impregnated paper powder layer to near 7, whereby the water-soluble compound of copper or zinc or copper and zinc is converted into water-insoluble hydroxide of copper or zinc or copper and zinc.

The paper powder layer impregnated with the hydroxide is moved along with the pH adjusting screen conveyer 75 and subjected to a pressing separation by a squeeze roller 82 mounted at the discharge end 81 of the pH adjusting screen conveyer 75, whereby a surplus aqueous solution of pH adjuster 83 is separated off. The hydroxide-impregnated paper powder layer 84 with the surplus aqueous solution of pH adjuster 83 separated off therefrom is transferred from the discharge end 81 of the pH adjusting screen conveyer 75 onto a pulverizing belt conveyer 57, where it is pulverized by a pulverizing roller 59 at a discharge end 58 of the pulverizing belt conveyer 57, and the resulting pulverized matter 60 is stored as a semi-finished product before dried in a semi-finished product tank 61.

Even in the present embodiment, the water-soluble compound of copper or zinc or copper and zinc is introduced along with citric acid into a compound dissolving and supplying tank 40, where it is heated by a heating device 42 and dissolved in water, as in the embodiment shown in FIG. 1. A dye is also introduced into a dye dissolving and supplying tank 46 where it is dissolved in water. A recovered surplus aqueous solution of compound is introduced from the surplus compound solution recovery tank 31 into a recovered-compound solution supply tank 47 by the feed pump 48. An aqueous solution of compound prepared by mixing together the aqueous solution of compound in the compound solution supply tank 40, the aqueous solution of the dye in the dye solution dissolving and supplying tank 46 and the recovered aqueous solution of compound in the recovered compound solution supply tank 47 by opening the on-off valves 43, 44 and 45 is supplied via the compound solution introducing pipe 9 and sprinkled from the compound solution sprinkling device 70 onto the paper powder moved to the compound solution sprinkling zone 69 of the screen conveyer 68, where it is mixed into the paper powder.

### EXAMPLE 1

100 Parts by weight of a paper powder having a particle size equal to or smaller than 0.35 mm was transported by the paper powder supplying belt conveyer and supplied to the screw conveyer. A solution of copper sulfate prepared by adding 200 parts by weight of water to a mixture of 22 parts by weight of copper sulfate and 2 parts by weight of citric acid and mixing them sufficiently was added to the paper powder transported within the screw conveyer by the screw, and the resulting mixture was mixed sufficiently. 322 parts by weight of the paper powder impregnated with the solution of copper sulfate was introduced into a solution of caustic soda made by dissolving 6 parts by weight of caustic soda in 500 parts of water, whereby the copper sulfate contained in the paper powder was converted into copper hydroxide. The paper powder impregnated with the precipitated copper hydroxide was separated, dehydrated, pulverized and then dried to provide a product as a deodorizer.

### EXAMPLE 2

This example is an example of the production of a deodorizer having copper hydroxide firmly adhered thereto. 100 Parts by weight of a paper powder having a particle size equal to or smaller than 0.35 mm was transported by the paper powder supplying belt conveyer and supplied to the screw conveyer. A solution of copper sulfate prepared by adding 200 parts by weight of water to a mixture of 22 parts by weight of copper sulfate, 2 parts by weight of sodium citrate and 12 parts by weight of carboxymethyl cellulose (CMC) and mixing them sufficiently was added to the paper powder transported within the screw conveyer by the screw, and the resulting mixture was mixed sufficiently. 334 Parts by weight of the paper powder carrying the carboxymethyl cellulose having copper ion adsorbed thereto was separated, washed by water, dehydrated, pulverized and dried to provide a product as a deodorizer.

A paper powder containing 0.2 to 1% by weight of copper can be used, for example, for a tissue, a toilet paper, a facial tissue, or a material having a sterilizing or deodorizing function and made using a dye. A paper powder containing 1 to 5% by weight of copper can be used, for example, for an absorbing article such as a granular excrement treating article, a pet sheet, a paper diaper, a breast pad and a urine-absorbing pad. A paper powder containing 5 to 10% by weight of copper can be used, for example, for an absorber for a mask, an absorbing article such as a paper towel, a filter for a cooling and heating device, a filter for a water purifier, a filter for circulation of a hot bath water, a filter for a cleaner, a wall paper, a shoji paper, and an absorbing article such as a paper diaper for an adult, a urine-absorbing pad, a deodorizer for an insert for a shoe and a deodorizer for a toilet.

The copper sulfate is used in the above-described examples, but copper chloride or zinc sulfate can be used. In the above-described examples, the amount of water used for the dissolving of the compound such as copper sulfate is smaller in order to decrease the amount of waste water generated, but the compound can be also dissolved in an amount of water as large as 1 to 2 litters. Further, in the above-described examples, the copper sulfate is neutralized, whereby copper hydroxide or copper dioxide is retained in the paper powder, but a copper compound to be retained can be basic copper sulfate or basic copper chloride. When carboxymethyl cellulose is used, copper sulfate, copper chloride, zinc sulfate or zinc chloride can be adsorbed, for example, to sodium carboxymethyl cellulose. Copper oxide, copper hydroxide, zinc sulfate or zinc hydroxide can be adsorbed to carboxylic acid-type carboxymethyl cellulose.

Referring to FIG. 3, a plastic waste is a recovered plastic waste resulting from the separation such as the classification of a paper diaper, and is pulverized into a particle size equal to or smaller than 5 mm and supplied to a metering hopper 1 for supplying the powder of the recovered plastic waste. A hopper 85 for supplying the powder of the recovered plastic waste is located on a transporting path of a belt conveyer device 86, so that the powder of the recovered plastic waste can be supplied to the belt conveyer device 86. In this embodiment, a metering hopper 87 for supplying a pulverized matter of a nonstandard waste of a paper diaper removed as a reject article is located on a transporting path of a belt conveyer device 88, so that the pulverized matter of the nonstandard paper diaper waste can be supplied to the belt conveyer device 88. In this embodiment, a transportation outlet 89 of the belt conveyer device 86 for supplying the powder of the recovered plastic waste and a transportation outlet 90 of the belt conveyer device 88 for supplying the pulverized matter of the nonstandard paper diaper waste are located at an introduction inlet of a first mixing belt conveyer device 91. The powder of the recovered plastic waste and the pulverized matter of the nonstandard paper diaper waste supplied from the belt conveyer device 86 and the belt conveyer device 88 to the first mixing belt conveyer device 91 are supplied from a transportation outlet 92 of the first mixing belt conveyer device 91 to a mixing hopper 93.

A mixture of the powder of the recovered plastic waste and the pulverized matter of the nonstandard paper diaper waste introduced into the mixing hopper 93 is supplied from a supply port 94 of the mixing hopper 93 to a second mixing belt conveyer device 95 and then supplied from the second mixing belt conveyer device 95 to a mixer 97 of a screw-type extrusion granulating device 96. The mixer 97 includes a screw by which the powder of the recovered plastic waste and the pulverized matter of the nonstandard paper diaper waste are mixed more uniformly. Water for the mixture to be granulated is supplied, for example, by mixing, into the mixture to be granulated, an aqueous solution of copper sulfate made by dissolving 2.4 grams of 5-hydrated copper sulfate in 10 litters of water. In the present embodiment, the mixer 97 is provided with an aqueous copper sulfate solution supply device 98, so that an aqueous solution of copper sulfate is injected from a nozzle 99 into the mixture of the powder of the recovered plastic waste and the pulverized matter of the nonstandard paper diaper waste, whereby the aqueous solution of copper sulfate supplied with granulating water is mixed into the mixture of the powder of the recovered plastic waste and the pulverized matter of the nonstandard paper diaper waste by the actions of an agitating blade, a screw and the like. The mixture of the powder of the recovered plastic waste and the pulverized matter of the nonstandard paper diaper waste containing the aqueous solution of copper sulfate mixed thereinto is mixed into a uniform or substantially uniform water-containing state. It is preferable that a mixing ratio of the aqueous solution of copper sulfate to the mixture of the powder of the recovered plastic waste and the pulverized matter of the nonstandard paper diaper waste is previously determined, so that a content of water in the mixture is a value suitable for the granulation. In the present embodiment, a disk pelleter is used for a granulating device and hence, the water content is adjusted to a value equal to or lower than 35% by weight. However, if the granulating device is a screw extrusion-type granulating device such as a meat chopper, then it is necessary to adjust the water content to a value higher than 35% by weight. If the water content is not covered by a liquid-state water-containing recovered highly water-absorptive resin, then an additional amount of water is supplied to the aqueous solution of copper sulfate, or water is supplied separately.

In the present embodiment, the mixture of the powder of the recovered plastic waste, the pulverized matter of the nonstandard paper diaper waste and the aqueous solution of copper sulfate is introduced into a disk pelleter 96 and extruded from a die 100 to provide a columnar granulated matter having a diameter of 4 mm and a length of 10 mm. A belt conveyer device 102 for transporting the granulated matter produced by the extrusion to a circular sieve 101 is mounted below the die 100 of the granulating device 96. In the present embodiment, a granulated matter supply port 103 of the granulated matter-transporting belt conveyer device 102 is located above the circular sieve. In the present embodiment, the opening size of the circular sieve 101 is about 3.5 mm smaller than 4 mm which is a grain size of grains of the granulated matter. The unsieved grains on the circular sieve 101 exit from an unsieved grain outlet 104 and are supplied to an unsieved grain-transporting belt conveyer 105 connected to the unsieved grain outlet 104.

A discharge end 106 of the unsieved grain-transporting belt conveyer 105 is located above and connected to a vibration-type grain size regulator 107. The vibration-type grain size regulator 107 is adapted to disintegrate grains of the granulated matter adhered to one another into individual grains by the vibration, and mounted in order to reduce the powdering during use to the utmost. The unsieved grains are supplied to the vibration-type grain size regulator 107 by the transporting belt conveyer device 105, where they are disintegrated into individual grains. On the other hand, sieved grains passed through the circular sieve 101 are transferred to a sieved grain-transporting belt conveyer 109 mounted at a sieved grain outlet 108 and returned from a discharge end 110 of the belt conveyer 109 to the second mixing belt conveyer 95 connected to the granulating device 96.

In the present embodiment, a sieve 111 having an opening size equal to or 4 mm is mounted at an outlet of the vibration-type grain size regulator 107. The grains of the granulated matter fed from the unsieved grain-transporting belt conveyer 105 to the vibration-type grain size regulator 107 are disintegrated into individual grains by the vibration of the vibration-type grain size regulator 107, and the resulting grains are each for forming a granular core and are fed to the sieve 111 at the outlet of the vibration-type grain size regulator 107. Below a sieved grain outlet (not shown) of the sieve 111, there is a sieved grain-transporting belt conveyer 113 having a discharge end 112 located on the mixture-transporting belt conveyer device 95, so that the sieved grains passed through the sieve 111 is returned to the second mixing belt conveyer device 95 connected to the granulating device 96 by the sieved grain-transporting belt conveyer 113.

In the vibration-type grain size regulator 107, the unsieved grains having a grain size equal to or larger than 4 mm are fed to the unsieved grain outlet 114. The unsieved grains fed to the unsieved grain outlet 114 is transported out of an unsieved grain-transporting belt conveyer 115 connected to the unsieved grain outlet 114 in order to ensure that surfaces of the unsieved grains are coated with a coating composition. An outlet of the unsieved grain-transporting belt conveyer 115 is provided on a feeder 118 of a vibration sieve-type first granulated matter coating device 117. In the present embodiment, a hopper for supplying a coating composition comprising a mixture of, for example, a paper powder having a particle size of 0.3 mm and a highly water-absorptive resin is connected to the first granulated matter coating device 117 through a coating composition supply passage 120, so that the coating composition is supplied to the vibration sieve-type first granulated matter coating device 117 by the coating composition supply passage 120.

The grains of the granulated matter disintegrated in the vibration-type grain size regulator 107 and having a grain size equal to or larger than 4 mm are grains each for forming a core and are transported out of the unsieved grain-transporting belt conveyer 115 connected to the unsieved grain outlet 111 and supplied to the feeder 118 of the first granulated matter coating device 117 connected to the unsieved grain-transporting belt conveyer 115. The grains each for forming the core are transported from the feeder 118 to the first granulated matter coating device 117, where a coating composition comprising a mixture of a paper powder having a particle size equal to or smaller than 0.3 mm and a highly water-absorptive resin is scattered onto such grains by the coating composition supply passage 120 connected to a coating composition supply hopper 119, whereby the surfaces of the grains are coated uniformly with the coating composition.

Any of the first, second and third granulated matter coating devices 117, 121 and 122 used in the present embodiment is a vibration-type granulated matter coating device of the same type. In the present embodiment, the three granulated matter coating devices 117, 121 and 122 are mounted in such a manner that they are connected in series to one another. More specifically, a discharge passage 125 from the first granulated matter coating device 117 is connected to the second granulated matter coating device 121, and a discharge passage 124 from the second granulated matter coating device 121 is connected to the third granulated matter coating device 122. In the first, second and third granulated matter coating devices 117,121 and 122, a coating composition comprising a mixture of a paper powder having a particle size equal to or smaller than 0.3 mm and a highly water-absorptive resin is adhered to the coated grains disintegrated in the vibration-type grain size regulator 107 and having the grain size equal to or larger than 4 mm, whereby a uniform coating layer is formed on the surface of each of cores.

In the present embodiment, the third granulated matter coating device 122 is provided with a water-spraying device 123, so that water is sprayed onto a surface of the coating layer of each of the coated grains from the spraying device 123 to prevent the fluffing or the like of the surface of the coating layer.

The coated grains sprayed on surfaces of their coating layers with the water in the third granulated matter coating device 122 are fed from an outlet 127 of the discharge passage 126 of the third granulated matter coating device 122, by a coated granulated matter transporting belt conveyer 128 mounted below the outlet 127, to a dryer 129 to which the coated granulated matter transporting belt conveyer 128 is connected. The coated grains coated by the coating composition and sprayed with the water are introduced to the dryer 129, e.g., such as a hot-air dryer, where they are dried. A dried-grain transporting belt conveyer device 131 is mounted below a dried-grain outlet 130 of the dryer 129. A discharge end of the dried and coated grain transporting belt conveyer device 131 is provided at an inlet 133 of a dried and coated grain accommodation tank 132, and the coated grains dried in the dryer 129 is transferred from the dried-grain outlet 130 of the dryer 129 to the dried and coated grain transporting belt conveyer device 131 and fed to the inlet 133 of the dried and coated grain accommodation tank 132 by the dried and coated grain transporting belt conveyer device 131.

In the present embodiment, the dried and coated grain accommodation tank 132 includes an exhaust portion 134 of a dust removing device (not shown). The dried and coated grains are taken out onto a dried and coated grain transporting belt conveyer device 136 by a dried and coated grain transporting chute 135 mounted below an outlet (not shown) of the dried and coated grain accommodation tank 132 and then fed to a grain-classifying circular vibration sieve system 137 including an upper-stage sieve having an opening size of, for example, 10 mm and a lower-stage sieve having an opening size of 4 mm, where they are separated into relatively coarse grains and relatively fine grains for size regulation. The size-regulated coated grains are a product for a granular excrement treating article and are discharged from a middle outlet (not shown) of the circular vibration sieve system 137. A product-transporting belt conveyer device 138 connected to the middle outlet is connected to an automatic packaging device 139, so that the product for the granular excrement treating article are fed to the automatic packaging device 139, where they are packaged automatically. A shipping belt conveyer device 140 is connected to the automatic packaging device 139, so that the packaged product is shipped from the shipping belt conveyer 140.

The apparatus in the present embodiment is constructed as described above and hence, a predetermined amount of a powder of a recovered plastic waste is placed into the metering hopper 85, and a predetermined amount of a pulverized matter of a nonstandard paper diaper waste is placed into the metering hopper 87.

Then, the belt conveyer device 86 for supplying the powder of the recovered plastic waste and the belt conveyer device 88 for supplying the pulverized matter of a nonstandard paper diaper waste are operated to supply the predetermined amounts of the powder of the recovered plastic waste and the pulverized matter of the nonstandard paper diaper waste respectively to the mixing hopper 93. The predetermined amounts of the powder of the recovered plastic waste and the pulverized matter of the nonstandard paper diaper waste are mixed together in the mixing hopper 93, and the resulting mixture is supplied to the mixture-transporting belt conveyer device 95 and fed to the previous mixer 97. In the present embodiment, in the previous mixer 97, an aqueous solution of copper sulfate is supplied from the feed nozzle 99 of the aqueous copper sulfate solution supply device 98 to the mixture of the powder of the recovered plastic waste and the pulverized matter of the nonstandard paper diaper waste, and they are mixed into a uniform or substantially uniform water-containing state, thereby forming a mixture to be granulated, which comprises the aqueous solution of copper sulfate mixed to the mixture of the powder of the recovered plastic waste and the pulverized matter of the nonstandard paper diaper waste. The water content of the mixture to be granulated is adjusted to substantially 30% by weight or less, when the granulating device used is a disk pelleter-type granulating device, or to substantially 30% by weight or more, when the granulating device used is a screw extrusion-type granulating device such as a meat chopper.

In the present embodiment, the granulating device is the screw extrusion-type granulating device, and the mixture to be granulated is extruded from a bore of the die 100 by the rotation of the screw. As a result, a columnar granulated matter having a section corresponding to a bore size of the die 100 cut into a predetermined length by a chopper, e.g., columnar grains of the granulated matter having an average length of 10 mm is obtained.

The granulated matter extruded from the die 100 of the granulating device 96 is fed to the circular sieve 101 by the granulating matter-transporting belt conveyer device 102 and subjected to a sieving treatment, and sieved grains having a grain size smaller than 4 mm are returned to the mixture-transporting belt conveyer device 95 by the sieved grain-transporting belt conveyer device 109. Unsieved grains having a grain size equal to or larger than 4 mm are fed to the vibration-type grain size regulator 107 by the unsieved grain-transporting belt conveyer device 105, where they are disintegrated into individual grains. The individual grains as a product for a granular excrement treating article, which have been produced by the disintegration of the grains having the grain size equal to or larger than 4 mm in the vibration-type grain size regulator 137, are fed from the product-transporting belt conveyer device 138 to the automatic packaging device 139, where they are packaged automatically. A product resulting from the automatic packaging in the automatic packaging device 139 is shipped from the shipping belt conveyer device 140.

In the present embodiment, additives, e.g., a substance having a sterilizing effect, a substance having a deodorizing effect, a substance having an adhesive effect or a coloring substance or a mixture of two or more of them is mixed into water and mixed into the coating layers of the grains of the coated granulated matter by spraying in the third granulated matter coating device 122. The grains of the coated granulated matter containing the coloring substance mixed thereto assumes a color such as white, yellow, green or blue color depending on the color of the coloring substance. In the present embodiment, a surfactant can be mixed into the coating layer portion by spraying onto the coating layer portion from the water-spraying device 123. When the components to be sprayed at the spraying step are two or more, spraying devices can be mounted for every component to be sprayed, if the mixing of the components is not preferred.

### EXAMPLE 3

30 Parts by weight of a pulverized matter of a nonstandard paper diaper waste, 50 parts by weight of a pulverized matter of a pulp-based non-woven fabric waste and 20 parts by weight of a pulverized matter of a rayon-based non-woven fabric waste were mixed together and supplied to a pulverizer (made by Horai, Co.), where they were pulverized through screen openings having an opening size of 4 mm. The resulting mixture of the nonstandard paper diaper waste, the pulverized matter of the pulp-based non-woven fabric waste and the pulverized matter of the rayon-based non-woven fabric waste was placed into a storage tank. The entire amount of an aqueous solution of copper sulfate prepared by dissolving 0.195 parts by weight of 5-hydrated copper sulfate in 20.48 parts by weight of water was added, while being sprayed, to the pulverized mixture removed from the storage tank by a quantitative feeder to provide a mixture to be granulated. The water content of the mixture was about 17% by weight. The mixture to be granulated was supplied to a disk pelleter (made by Fuji Powder, Co.) of a disk-type extrusion granulating device, where it was extruded and granulated by a rotary roller through a die having a thickness of 25 mm and a bore size of 4 mm to provide grains having a diameter of 4 mm and a length of 10 mm (and having a water content of 10 to 20% by weight). The columnar grains resulting from the extrusion and granulation were supplied onto an upper-stage sieve of a circular sieving device (made by Dultone, Co.) including the upper-stage sieve having an opening size of 10 mm and a lower-stage sieve having an opening size of 4 mm, where grains having a grain size larger than 10 mm and grains having a grain size smaller than 4 mm were sieved off. The grains having the grain size larger than 10 mm and the grains having the grain size smaller than 4 mm, which were sieved off, were fed to a pulverized matter storage tank and subjected again to the granulation. The grains remaining after the sieving-off of the grains having the grain size larger than 10 mm and the grains having the grain size smaller than 4 mm were supplied to a circular sieving device (made by Dultone, Co.), where water was sprayed onto the grains on a sieve thereof (having an opening size of 2 mm). The grains sprayed with the water had a water content of 27 to 30% by weight, and was supplied onto a vibrating plate of a circular coating device (made by Dultone, Co.) by a belt conveyer. In this example, a coating composition was scattered in the circular coating device, so that it was of 17 parts by weight (based on a dried state), and the grains were of 87 parts by weight (based on a dried state). In this example, coating steps using the coating composition were carried out with five circular coating devices arranged in series. At the final coating step, water was sprayed onto the coated grains subjected to the coating treatment in order to smooth the surfaces of the coated grains. The coated grains sprayed with the water were fed to a hot-air dryer, where they were dried at a temperature equal to or higher than 80° C. by hot air, until the water content was equal to or lower than 12% by weight. The coated grains dried by the hot air were temporarily placed into a commodity hopper, where they were thermally conditioned to room temperature. The dried coated grains thermally conditioned to the room temperature were supplied to an upper-stage sieve of a circular sieving device (made by Dultone, Co.) comprising the upper-stage sieve having an opening size of 10 mm and a lower-stage sieve having an opening size of 4 mm, where they were sieved into relatively large grains and relatively fine grain to meet a commodity standard, thereby producing mainly grains having a diameter of 4.2 mm and a length of 10 mm. These grains were gathered and fed to a packaging device, where they were bagged to provide a sand article for a cat toilet.

The sand article for the cat toilet fabricated in this example contains copper in a content of 0.05 parts by weight per 100 parts by weight of granular cores. The sand article for the cat toilet fabricated in this example was used for the excretion by a cat, but it was not found that copper sulfate was adhered to a cat's body after urination. In the sand article for the cat toilet fabricated in this example, the sand grains were agglomerated in the same manner as in the conventional sand article for a cat toilet, and it was easy to distinguish a wetted area.

The sand article for the cat toilet fabricated in this example comprises copper sulfate contained in granular cores, and when ammonia water diluted to 20 times was added to the sand article at a temperature of 35° C. in a room and as a result, the sand article exhibited very good water-absorbency and deodorization, whereby no ammonia odor was felt within the room. The sand article for the cat toilet was tested, laid at a depth of 7 cm in a commercially available cat toilet (W430×D310×H135) for a test, and as a result, a cat tended to gradually urinated and defecated without problem as it was becoming accustomed to do so. After use, it was easy to discard the portion wetted by the urine, and urine's odor was little felt within the room.

In this example, the coating composition was prepared by mixing (1) 55 parts by weight of a paper powder made by pulverizing a fine powdery fluff containing a water-absorptive resin generated in a sanitary good-producing factory into a particle size equal to or smaller than 0.3 mm by a pulverizer (made by Turbo Industries, Co.), (2) 20 parts by weight of a powder made by pulverizing a highly water-absorptive resin, Haimosabu HS-1100((a trade name) made by Haimo, Co.), having an adhesive function into a particle size equal to or smaller than 40 μm by a pulverizer (made by Turbo Industries, Co.), (3) 16 parts by weight of a powder made by pulverizing a highly water-absorptive resin, AP-900S ((a trade name) made by Mitsubishi Chemical, Co.) into a particle size equal to or smaller than 40 μm by a pulverizer (made by Turbo Industries, Co.), and (4) 2 parts by weight of a powder made by pulverizing acrylamide (made by Diaflock, Co.) into a particle size equal to or smaller than 40 μm by a pulverizer (made by Turbo Industries, Co.).

### EXAMPLE 4

65 Parts by weight of a nonstandard paper diaper waste was pulverized, passed through screen openings having an opening size of 4 mm, and the resulting pulverized matter was placed into a storage tank and then removed by a screw feeder. 35 Parts of used tea leaves having a water content of 70% by weight was mixed with the pulverized matter of the paper diaper waste removed by the screw feeder. In this embodiment, the entire amount of an aqueous solution of copper sulfate prepared by adding and dissolving 0.15 parts by weight of 5-hydrated copper sulfate in 6 parts by weight of water was mixed into the resulting mixture to prepare a mixture to be granulated. This mixture had a water content of 29% by weight. This mixture was supplied to a disk pelleter (made by Fuji Powder, Co.) of a disk-type extrusion granulating device, where it was extruded at a thickness of 25 mm through a die having a bore size of 4 mm by a rotary roller to form grains having a diameter of 4 mm and a length of 10 mm by granulation. The columnar grains formed by the extruding granulation were supplied to an upper-stage sieve of a circular sieving device (made by Dultone, Co.) comprising the upper-stage sieve having an opening size of 10 mm and a lower-stage sieve having an opening size of 4 mm, where grains having a grain size larger than 10 mm and grains having a grain size smaller than 4 mm were sieved off. The grains remaining after sieving-off of the grains having the grain size larger than 10 mm and the grains having the grain size smaller than 4 mm were supplied to a circular sieving device (made by Dultone, Co.), where water was sprayed onto the grains on its sieve (having an opening size of 2 mm). The grains sprayed with the water had a water content of 27 to 30% by weight and was supplied onto a vibrating plate of a circular coating device (made by Dultone, Co.) by a belt conveyer. In this example, a coating composition was scattered in the circular coating device, so that it was of 17 parts by weight (based on a dried state), and the grains were of 87 parts by weight (based on a dried state). In this example, coating steps using the coating composition were carried out with five circular coating devices arranged in series. At the final coating step, water was sprayed onto the coated grains subjected to the coating treatment in order to smooth the surfaces of the coated grains. The coated grains sprayed with the water were fed to a hot-air dryer, where they were dried at a temperature equal to or higher than 80° C. by hot air, until the water content was equal to or lower than 12% by weight. The coated grains dried by the hot air were temporarily placed into a commodity hopper, where they were thermally conditioned to room temperature. The dried coated grains thermally conditioned to the room temperature were supplied to an upper-stage sieve having an opening size of 10 mm included in a circular sieving device (made by Dultone, Co.) further including a lower-stage sieve having an opening size of 4 mm, where they were sieved into relatively large grains and relatively fine grain to meet a commodity standard, thereby producing mainly grains having a diameter of 4.2 mm and a length of 10 mm. These grains were gathered and fed to a packaging device, where they were bagged to provide a sand article for a cat toilet.

The sand article for the cat toilet fabricated in this example contains copper in a content of 0.05 parts by weight per 100 parts by weight of granular cores. The sand article for the cat toilet fabricated in this example was used for the excretion by a cat, but the deposition of copper sulfate on a cat's body after urination was not observed. In the sand article for the cat toilet fabricated in this example, the sand grains were agglomerated in the same manner as in the conventional sand article for a cat toilet, and it was easy to distinguish a wetted area.

The sand article for the cat toilet fabricated in this example comprises copper sulfate contained in granular cores, and ammonia water diluted to 20 times was added to the sand article at a temperature of 35° C. in a room and the result showed that the sand article exhibited very good water-absorbency and deodorization, whereby no ammonia odor was felt within the room. The sand article for the cat toilet was tested, laid at a depth of 7 cm in a commercially available cat toilet (W430×D310×H135) for a test, and as a result, a cat tended to gradually urinated and defecated without problem as it was becoming accustomed to do so. After use, it was easy to discard the portion wetted by the urine, and a urine's odor was little felt within the room.

In this example, the coating composition was prepared by mixing (1) 60 parts by weight of a paper powder made by pulverizing a fine-powdery fluff containing a water-absorptive resin generated in a sanitary good-producing factory into a particle size equal to or smaller than 0.3 mm by a pulverizer (made by Turbo Industries, Co.), (2) 38 parts by weight of a powder made by pulverizing a highly water-absorptive resin, Haimosabu HS-1100[(a trade name) made by Haimo, Co.], having an adhesive function into a particle size equal to or smaller than 40 μm by a pulverizer (made by Turbo Industries, Co.).

### EXAMPLE 5

70 Parts by weight of a skin of a cabbage having a water content of 80% by weight was supplied to a wet pulverizer (made by Horai, Co.) and pulverized through screen openings having an opening size of 5 mm, and the resulting pulverized matter was placed into a ribbon mixer. 60 Parts by weight of an inorganic fiber paper (containing titanium) discharged from a paper-making factory was supplied to a dry pulverizer (made Horai, Co.) and pulverized through screen openings having an opening size of 3 mm, and the resulting pulverized matter of the inorganic fiber paper was placed into the ribbon mixer containing the pulverized matter of the cabbage skin, where they were mixed together. Then, 3 parts by weight of acrylamide (made by Diaflock, Co.) was added to and further mixed with this mixture. The entire amount of an aqueous solution of copper sulfate prepared by dissolving 0.15 parts by weight of copper sulfate in 6 parts by weight of water was added to the resulting mixture to prepare a mixture to be granulated, containing the aqueous solution of copper sulfate. The mixture to be granulated had a water content of 48% by weight. This mixture was supplied to a meat chopper (made by Hiraga Kosakujo, Co.) and extruded by a screw through a die having a thickness of 25 mm and a bore size of 4 mm and thus granulated into grains having a diameter of 4 mm and a length of 10 mm (and having a water content of about 23 to 28% by weight). The columnar grains made by the extrusion and granulation were supplied onto an upper-stage sieve having an opening sizes of 10 mm and included in a circular sieving device (made by Dultone, Co.) further including a lower-stage sieve having an opening size of 4 mm, whereby grains having a grain size larger than 10 mm and grains having a grain size smaller than 4 mm were removed by sieving. The grains having the grain sizes larger than 10 mm and smaller than 4 mm removed here were fed to a grain storage tank and supplied again for the granulation. The grains remaining after removal of the grain grains having the grain size larger than 10 mm and the grains having the grain size smaller than 4 mm by the sieving were supplied to a circular sieving device (made by Dultone, Co.), where water was sprayed to the grain on a sieve (having an opening size of 2 mm). The grains sprayed with the water had a water content of 27 to 30% by weight and was supplied onto a vibration plate of a circular coating device (made by Dultone, Co.) by a belt conveyer. In this example, a coating composition was scattered on the grains in the circulating coating device, so that the grains were of 87 parts by weight (based on a dry state) and the coating composition was of 17 parts by weight (based on a dry state). In this example, coating steps using the coating composition was carried out with five circular coating devices arranged in series. At the final coating step, water was sprayed onto the coated grains resulting from the coating treatment in order to smooth the surfaces of the coated grains. The coated grains sprayed with the water were fed to a hot-air dryer, where they were dried at a temperature equal to or higher than 80° C. by hot air until the water content reached equal to or lower than 12% by weight. The coated grains dried by the hot air were temporarily placed into a commodity hopper, where they were thermally conditioned to room temperature. The dried coated grains thermally conditioned to the room temperature were supplied onto an upper-stage sieve having an opening size included in a circular sieving device (made by Dultone, Co.) further including a lower-stage sieve having an opening size of 4 mm, where they were sieved into relatively large grains and relatively fine grains to meet a standard for commodity, thereby providing mainly grains having a diameter of 4.2 mm and a length of 10 mm. These grains were gathered and fed to a packaging device, where they were bagged to provide a sand article for a cat toilet.

The sand article for the cat toilet fabricated in this example comprises 0.05 parts by weight of copper contained in 100 parts by weight of granular cores, as in Examples 1 and 2. The sand article for the cat's toilet fabricated in this example was used for excretion of a cat, but the deposition of copper sulfate to a cat's body after urination was not observed. The grains of the sand article for the cat toilet fabricated in this example were bound and coagulated, as were a conventional sand article for a cat toilet, and it was easy to distinguish a wefted area.

The sand article for the cat toilet fabricated in this example comprises the copper sulfate contained in the granular cores, and 50 ml of ammonia water diluted 20 times was added to the sand article at a temperature of 35° C. in a room, and the result showed that the sand article exhibited very good water-absorbency and deodorization, whereby no ammonia odor was felt within the room. The sand article for the cat toilet was tested, laid at a depth of 7 cm in a commercially available cat toilet (W430×D310×H135) for a test, and as a result, a cat tended to gradually urinated and defecated without problem as it was becoming accustomed to do so. After use, it was easy to discard the portion wetted by the urine, and a urine's odor was little felt within the room.

In this example, the coating composition was prepared by mixing (1) 55 parts by weight of a paper powder made by pulverizing a fine-powdery fluff containing a water-absorptive resin generated in a sanitary good-producing factory into a particle size equal to or smaller than 0.3 mm by a pulverizer (made by Turbo Industries, Co.), (2) 20 parts by weight of a powder made by pulverizing a highly water-absorptive resin, Haimosabu HS-1100[(a trade name) made by Haimo, Co.], having an adhesive function into a particle size equal to or smaller than 40 μm by a pulverizer (made by Turbo Industries, Co.), (3) 16 parts by weight of a powder made by pulverizing a highly water-absorptive resin AP-900S [(a trade name) made by Mitsubishi Chemistry, Co.] into a particle size equal to or smaller than 40 μm by a pulverizer (made by Turbo Industries, Co.), and (4) 2 parts by weight of a powder made by pulverizing acrylamide (made by Diaflock, Co.) into a particle size equal to or smaller than 40 μm by a pulverizer (made by Turbo Industries, Co.).

### EXAMPLE 6

60 Parts by weight of kitchen garbage such as cooking refuses, food residues and leftovers from a dining room was supplied to a wet pulverizer (made by Horai, Co.) and pulverized through screen openings having an opening size of 3 mm (sewage generated during pulverization was subjected to an activated sludge treatment). The pulverized matter of the kitchen garbage was placed into a ribbon mixer. 10 Parts by weight of white carbon was placed into the ribbon mixer with the pulverized matter of the kitchen garbage contained therein, where it was mixed with the pulverized matter of the kitchen garbage. 80 Parts by weight of used tea leaves (such as green tea, oolong tea, red tea and a blend tea(adlay tea and barley tea) having a water content of 70% by weight and 3 parts by weight of sorbic acid were added to and mixed with the resulting mixture. On the other hand, 60 parts by weight of a classification product rich in plastic waste from a paper diaper waste was supplied to a dry pulverizer (made by Horai, Co.) and pulverized through screen openings having an opening size of 3 mm. The pulverized matter of the classification product from the paper diaper waste was placed into the ribbon mixer, where it was mixed. The resulting mixture had a water content of 42% by weight. In the ribbon mixer, the entire amount of an aqueous solution of copper sulfate prepared by dissolving 0.25 parts by weight of 5-hydrated copper sulfate in 5 parts by weight of water was added to and mixed with the mixture to provide a mixture to be granulated. The mixture to be granulated had a water content of 43% by weight. The mixture to be granulated was removed from the ribbon mixer by a screw feeder and supplied to a meat chopper (made by Hiraga, Seisakujo, Co.) of a screw-extrusion granulating device by this screw feeder, where it was extruded and granulated by a screw through a die having a thickness of 25 mm and a bore size of 4 mm to provide grains having a diameter of 4 mm and a length of 10 mm (and having a water content of 18% to 20% by weight). The columnar grains made by the extrusion and granulation were supplied onto an upper-stage sieve having an opening size of 10 mm of a circular sieving device (made by Dultone, Co.) further including a lower-stage sieve having an opening size of 4 mm, whereby grains having a grain size equal to or larger than 10 mm and grains having a grain size equal to or smaller than 4 mm were removed by sieving. The grains having the grain size equal to or larger than 10 mm and the grains having the grain size equal to or smaller than 4 mm, which were removed, were fed to a grain storage tank and subjected again to the granulation. The grains remaining after removal of the grains having the grain size equal to or larger than 10 mm and the grains having the grain size equal to or smaller than 4 mm by sieving were supplied to a circular sieving device (made by Dultone, Co.), where water was sprayed onto the grains on a sieve (having an opening size of 2 mm). The grains sprayed with the water had a water content of 27% to 30% by weight and were supplied onto a vibration plate of a circular coating device (made by Dultone, Co.) by a belt conveyer. In this example, a coating composition was scattered to the grains in the circular coating device, so that the grains were of 87 parts by weight (based on a dried state) and the coating composition was of 17 parts by weight (based on a dried state). In this example, coating steps using the coating composition were carried out with five circular coating devices arranged in series. At the final coating step, water was sprayed onto the coated grains subjected to the coating treatment in order to smooth the surfaces of the coated grains. The coated grains sprayed with the water were fed to a hot-air drier, where they were dried at a temperature of 80° C. or more by hot air, until the water content reached equal to or lower than 12% by weight. The coated grains dried by the hot air were temporarily placed into a commodity hopper and thermally conditioned to room temperature. The coated grains thermally conditioned to the room temperature were supplied onto an upper-stage sieve having an opening size of 10 mm included in a circular sieving device (made by Dultone, Co.) further including a lower-stage sieve having an opening size of 4 mm, where they were sieved into relatively large grains and relatively fine grains to meet a standard for commodity, thereby providing mainly grains having a diameter of 4.2 mm and a length of 10 mm were obtained. These grains were gathered and fed to a packaging device, where they were bagged to provide a sand article for a cat toilet.

The sand article for the cat toilet fabricated in this example comprises 0.05 parts by weight of copper contained in 100 parts by weight of granular cores, as in Examples 1 to 3. The sand article for the cat toilet fabricated in this example was used for excretion of a cat, and as a result, the deposition of copper sulfate to a cat's body after urination was not observed. The grains of the sand article for the cat toilet fabricated in this example were bound and coagulated after urination, as were a conventional sand article for a cat toilet, and it was easy to distinguish a wefted area.

The sand article for the cat toilet fabricated in this example comprises the copper sulfate contained in the granular cores, and 50 ml of ammonia water diluted 20 times was added to the sand article at a temperature of 35° C. in a room, and the result showed that the sand article exhibited very good water-absorbency and deodorization, whereby no ammonia odor was felt within the room. The sand article for the cat toilet was tested, laid at a depth of 7 cm in a commercially available cat toilet (W430×D310×H135) for a test, and as a result, a cat tended to gradually urinated and defecated without problem as it was becoming accustomed to do so. After use, it was easy to discard the portion wetted by the urine, and a urine's odor was little felt within the room.

In this example, the coating composition was prepared by mixing (1) 55 parts by weight of a paper powder made by pulverizing a fine-powdery fluff containing a water-absorptive resin generated in a sanitary good-producing factory into a particle size equal to or smaller than 0.3 mm by a pulverizer (made by Turbo Industries, Co.), (2) 20 parts by weight of a powder made by pulverizing a highly water-absorptive resin, Haimosabu HS-1100[(a trade name) made by Haimo, Co.], having an adhesive function into a particle size equal to or smaller than 40 μm by a pulverizer (made by Turbo Industries, Co.), (3) 13 parts by weight of a powder made by pulverizing a highly water-absorptive resin AP-900S [(a trade name) made by Mitsubishi Chemistry, Co.] into a particle size equal to or smaller than 40 μm by a pulverizer (made by Turbo Industries, Co.), (4) 10 parts by weight of a powder made by pulverizing acrylamide (made by Diaflock, Co.) into a particle size equal to or smaller than 40 μm by a pulverizer (made by Turbo Industries, Co.), and (5) 5 parts by weight of a green choke powder (made by Nippon Hakuboku Industries, Co.) having a particle size equal to or smaller than 270 meshes (an opening size of a sieve being of 53 μm).

### EXAMPLE 7

50 Parts by weight of buff powder discarded from a printing factory, 30 parts by weight of Pinkasu, i.e., a punching refuse from a printing factory and 20 parts by weight of a print refuse of a laminate paper were supplied to a dry pulverizer (made by Horai, Co.) and pulverized through screen openings having an opening size of 4 mm. The entire amount of an aqueous solution of copper sulfate made by dissolving 0.2 parts by weight of 5-hydrated copper sulfate in 5 parts of water was added to the pulverized mixture, and a water-containing and water-absorptive liquid resin recovered was sprayed onto the mixture, so that the water content of the mixture amounted to 17% by weight. The resulting mixture was supplied to a disk pelleter (made by Fuji Powder, Co.) of a disk-type extrusion granulating device by a screw feeder. In the supplying of the mixture to the disk pelleter, a die of the disk pelleter had a thickness of 25 mm and a bore size of 4 mm. The mixture supplied to the disk pelleter was extruded and granulated through the die by a rotary roller to provide grains having a diameter of 4 mm and a length of 10 mm. The columnar grains made by the extrusion and granulation were supplied to an upper-stage sieve having an opening size of 10 mm included in a circular sieving device (made by Dultone, Co.) further including a lower-stage sieve having an opening size of 4 mm, whereby grains having a grain size equal to or larger than 10 mm and grains having a grain size equal to or smaller than 4 mm were removed by sieving. The grains having the grain size equal to or larger than 10 mm and the grains having the grain size equal to or smaller than 4 mm removed by sieving were fed to a grain storage tank and subjected again to the granulation. Grains remaining after the removal of the grains having the grain size equal to or larger than 10 mm and the grains having the grain size equal to or smaller than 4 mm were supplied to a circular sieving device (made by Dultone, Co.), where water was sprayed onto the grains on a sieve (having an opening size of 2 mm). The grains sprayed with the water had a water content of 27 to 30% by weight, and was supplied onto a vibrating plate of a circular coating device (made by Dultone, Co.) by a belt conveyer. In this example, a coating composition was scattered in the circular coating device, so that it was of 17 parts by weight (based on a dried state), and the grains were of 87 parts by weight (based on a dried state). In this example, coating steps using the coating composition were carried out with five circular coating devices arranged in series. At the final coating step, water was sprayed onto the coated grains subjected to the coating treatment in order to smooth the surfaces of the coated grains. The coated grains sprayed with the water were fed to a hot-air dryer, where they were dried at a temperature equal to or higher than 80° C. by hot air, until the water content was equal to or lower than 12% by weight. The coated grains dried by the hot air were temporarily placed into a commodity hopper, where they were thermally conditioned to room temperature. The dried coated grains thermally conditioned to the room temperature were supplied to an upper-stage sieve having an opening size of 10 mm included in a circular sieving device (made by Dultone, Co.) further including a lower-stage sieve having an opening size of 4 mm, where they were sieved into relatively large grains and relatively fine grain to meet a commodity standard, thereby producing mainly grains having a diameter of 4.2 mm and a length of 10 mm. These grains were gathered and fed to a packaging device, where they were bagged to provide a sand article for a cat toilet.

The sand article for the cat toilet fabricated in this example contains copper in a content of 0.05 parts by weight per 100 parts by weight of granular cores, as in Examples 1 to 4. The sand article for the cat toilet fabricated in this example was used for the excretion by a cat, but the deposition of copper sulfate on a cat's body after urination was not observed. In the sand article for the cat toilet fabricated in this example, the sand grains were bound and agglomerated in the same manner as in the conventional sand article for a cat toilet, and it was easy to distinguish a wetted area.

The sand article for the cat toilet fabricated in this example comprises copper sulfate contained in granular cores, and when ammonia water diluted to 20 times was added to the sand article at a temperature of 35° C. in a room and as a result, the sand article exhibited very good water-absorbency and deodorization, whereby no ammonia odor was felt within the room. The sand article for the cat toilet was tested, laid at a depth of 7 cm in a commercially available cat toilet (W430×D310×H135) for a test, and as a result, a cat tended to gradually urinated and defecated without problem as it was becoming accustomed to do so. After use, it was easy to discard the portion wetted by the urine, and urine's odor was little felt within the room.

In this example, the coating composition was prepared by mixing (1) 60 parts by weight of a paper powder made by pulverizing a fine powdery fluff containing a water-absorptive resin generated in a sanitary good-producing factory into a particle size equal to or smaller than 0.3 mm by a pulverizer (made by Turbo Industries, Co.), (2) 20 parts by weight of a powder made by pulverizing a highly water-absorptive resin, Haimosabu HS-1100((a trade name) made by Haimo, Co.), having an adhesive function into a particle size equal to or smaller than 40 μm by a pulverizer (made by Turbo Industries, Co.), (3) 13 parts by weight of a powder made by pulverizing tapioca alpha starch (made by Matsutani Chemical Industries, Co.) into a particle size equal to or smaller than 40 μm by a pulverizer (made by Turbo Industries, Co.), and (4) 10 parts by weight of a powder made by pulverizing acrylamide (made by Diaflock, Co.) into a particle size equal to or smaller than 40 μm by a pulverizer (made. by Turbo Industries, Co.).

In these examples, the content of copper contained in each of the granular cores is 0.01 part by weight per 100 parts by weight of the granular core, but may be smaller than 0.01 part by weight per 100 parts by weight of the granular core, or larger than 0.01 part by weight per 100 parts by weight of the granular core. Even a water-soluble copper or zinc compound having a deodorizing function other than zinc sulfate such as an aqueous solution of copper sulfate can be likewise used. For a water-insoluble copper or zinc compound having a deodorizing function such as copper hydroxide, a suspension made by suspending the water-insoluble copper or zinc compound in water can be sprayed or scattered onto the surfaces of the grains, whereby the water-insoluble copper or zinc compound having the deodorizing function such as copper hydroxide can be deposited on the surfaces of the grains.

### INDUSTRIAL APPLICABILITY

The present invention is directed to a material having a deodorizing function, which includes a paper powder having a particle size equal to or smaller than 0.35 and contains a copper or zinc compound or copper and zinc compounds retained and carried therein. Therefore, it is possible to provide a material having a deodorizing function, in which a paper powder of a waste can be used as a retaining material, and the cost is low, as compared with the conventional material.

The present invention is directed to a process for producing a material having a deodorizing function, which comprises mixing an aqueous solution of a copper or zinc compound or copper and zinc compounds into a paper powder, particularly, a paper powder having a water absorbency adjusted to a suitable level, mixing the mixture into an aqueous solution of a pH adjuster to form an insoluble basic compound or insoluble hydroxide of copper or zinc or copper and zinc, allowing the insoluble basic compound or insoluble hydroxide to be retained in the paper powder, drying the resulting mixture and then pulverizing the mixture into a particle size equal to or smaller than 0.35 mm. Therefore, it is possible to produce a material having a deodorizing function at a small number of steps of impregnating the paper power with an aqueous solution of a copper or zinc compound or copper and zinc compounds and converting the copper or zinc compound or copper and zinc compounds into hydroxide and in a simple operation as compared with the conventional producing process.

In addition, the present invention is directed to a granular excrement treating article, which is formed into a coated granular material and which comprises a granular core portion formed into a granular shape with a grain size equal to or larger than 1 mm and containing a power of an organic waste, a smaller amount of a material having an adhesive property than the power of the organic waste, an additive and a copper compound or a zinc compound or a metal compound of two or more metals having a deodorizing function, and a coating layer portion covering at least a portion of a surface of the granular core portion and containing a paper powder and a material having an adhesive property. Therefore, an offensive odor of excrement after use of the granular excrement treating article can be removed by the copper or zinc compound having the deodorizing function, and the coating layer portion cannot be adhered to an animal's body and cannot stain the surroundings after use of the granular excrement treating article, and hence, the surrounding environment for breeding an animal can be kept clean. Moreover, the present invention produces a granular excrement treating article using a plastic waste and/or an organic water as a starting material(s) and thus, the application of materials discarded as wastes is opened up, enabling an effective utilization of a waste, which provides a large effect.

1. A material having a deodorizing function, characterized in that it comprises a paper powder, and a copper compound or a zinc compound or copper and zinc compounds contained and retained in said paper powder.

2. A material having a deodorizing function, characterized in that it comprises a paper powder, carboxymethyl cellulose secured to said paper powder, and a copper compound or a zinc compound or copper and zinc compounds contained and retained in said carboxymethyl cellulose.

3. A material having a deodorizing function, characterized in that it comprises a paper powder, and a water-insoluble complex or a basic compound or hydroxide compound of copper or zinc, or water-insoluble complexes or basic compounds or hydroxide compounds of copper and zinc, said water-insoluble complex or a basic compound or hydroxide compound being retained in said paper powder.

4. A material having a deodorizing function, characterized in that it comprises a paper powder, carboxymethyl cellulose secured to said paper powder, and a water-insoluble complex or a basic compound or hydroxide compound of copper or zinc, or water-insoluble complexes or basic compounds or hydroxide compounds of copper and zinc, said water-insoluble complex or a basic compound or hydroxide compound being contained and retained in said carboxymethyl cellulose.

5. A granular excrement treating article formed into a granular shape with a grain size equal to or larger than 1 mm, characterized in that it comprises a powder of an organic waste, a smaller amount of a material having an adhesive property than the amount of said powder of the organic waste, and a paper powder in which a copper compound or a zinc compound or copper and zinc compounds having a deodorizing function is or are retained.

6. A granular excrement treating article formed into a granular shape with a grain size equal to or larger than 1 mm, characterized in that it comprises a powder of an organic waste, a smaller amount of a material having an adhesive property than the amount of said powder of the organic waste, an additive, and a paper powder in which a copper compound or a zinc compound or copper and zinc compounds having a deodorizing function is or are retained.

7. A granular excrement treating article formed into a granular shape with a grain size equal to or larger than 1 mm, characterized in that it comprises a powder of an organic waste, a smaller amount of a material having an adhesive property than the amount of said powder of the organic waste, and a paper powder to which carboxymethyl cellulose is secured, a copper compound or a zinc compound or copper and zinc compounds having a deodorizing function being bonded or adhered to the carboxymethyl cellulose.

8. A granular excrement treating article formed into a granular shape with a grain size equal to or larger than 1 mm, characterized in that it comprises a powder of an organic waste, a smaller amount of a material having an adhesive property than the amount of said powder of the organic waste, an additive, and a paper powder to which carboxymethyl cellulose is secured, a copper compound or a zinc compound or copper and zinc compounds having a deodorizing function being bonded or adhered to the carboxymethyl cellulose.

9. A granular excrement treating article formed into a coated granulated material and comprising a granular core portion and a coating layer portion covering said granular core portion, characterized in that said granular core portion is formed into a granular shape with a grain size equal to or larger than 1 mm and containing a powder of an organic waste, a smaller amount of a material having an adhesive property than the amount of said powder of the organic waste, and a paper powder in which a copper compound or a zinc compound or copper and zinc compounds having a deodorizing function is or are retained, and said coating layer portion is formed to cover at least a portion of a surface of said granular core portion and to contain a paper powder and a material having an adhesive property.

10. A granular excrement treating article formed into a coated granulated material and comprising a granular core portion and a coating layer portion covering said granular core portion, characterized in that said granular core portion is formed into a granular shape with a grain size equal to or larger than 1 mm and containing a powder of an organic waste, a smaller amount of a material having an adhesive property than the amount of said powder of the organic waste, an additive, and a paper powder in which a copper compound or a zinc compound or copper and zinc compounds having a deodorizing function is or are retained, and said coating layer portion is formed to cover at least a portion of a surface of said granular core portion and to contain a paper powder and a material having an adhesive property.

11. A granular excrement treating article formed into a coated granulated material and comprising a granular core portion and a coating layer portion covering said granular core portion, characterized in that said granular core portion is formed into a granular shape with a grain size equal to or larger than 1 mm and containing a powder of an organic waste, and a smaller amount of a material having an adhesive property than the amount of said powder of the organic waste; said coating layer portion is formed to cover said granular core portion and to contain a paper powder and a material having an adhesive property; and a paper powder retaining a copper compound or a zinc compound or a copper compound and a zinc compound having a deodorizing function therein is contained in said granular core portion, between said granular core portion and said coating layer portion, in at least a portion of said coating layer portion or in at least a portion of a surface of said coating layer portion, or between said granular core portion and said coating layer portion, as well as in at least a portion of said coating layer portion or in at least a portion of a surface of said coating layer portion, or in at least a portion of said coating layer portion and in at least a portion of a surface of said coating layer portion.

12. A granular excrement treating article formed into a coated granulated material and comprising a granular core portion and a coating layer portion covering said granular core portion, characterized in that said granular core portion is formed into a granular shape with a grain size equal to or larger than 1 mm and containing a powder of an organic waste, and a smaller amount of a material having an adhesive property than the amount of said powder of the organic waste; said coating layer portion is formed to cover said granular core portion and to contain a paper powder, a material having an adhesive property, and an additive; and a paper powder retaining a copper compound or a zinc compound or a copper compound and a zinc compound having a deodorizing function therein and an additive are contained in said granular core portion, between said granular core portion and said coating layer portion, in at least a portion of said coating layer portion or in at least a portion of a surface of said coating layer portion, or between said granular core portion and said coating layer portion, as well as in at least a portion of said coating layer portion or in at least a portion of a surface of said coating layer portion, or in at least a portion of said coating layer portion and in at least a portion of a surface of said coating layer portion.

13. A granular excrement treating article formed into a coated granulated material and comprising a granular core portion and a coating layer portion covering said granular core portion, characterized in that said granular core portion is formed into a granular shape with a grain size equal to or larger than 1 mm and containing a powder of an organic waste, a smaller amount of a material having an adhesive property than the amount of said powder of the organic waste, and an additive; said coating layer portion is formed of a mixtureof a paper powder and a material having an adhesive property to cover said granular core portion; and a paper powder retaining a copper compound or a zinc compound or a copper compound and a zinc compound having a deodorizing function therein is contained in said granular core portion, between said granular core portion and said coating layer portion, in at least a portion of said coating layer portion or in at least a portion of a surface of said coating layer portion, or between said granular core portion and said coating layer portion, as well as in at least a portion of said coating layer portion or in at least a portion of a surface of said coating layer portion, or in at least a portion of said coating layer portion and in at least a portion of a surface of said coating layer portion.

14. A granular excrement treating article formed into a coated granulated material and comprising a granular core portion and a coating layer portion covering said granular core portion, characterized in that said granular core portion is formed into a granular shape with a grain size equal to or larger than 1 mm and containing a powder of an organic waste, a smaller amount of a material having an adhesive property than the amount of said powder of the organic waste, and an additive; said coating layer portion is formed of a mixture of a paper powder and a material having an adhesive property to cover said granular core portion; and a paper powder accreting carboxymethyl cellulose in which a copper compound or a zinc compound or a copper compound and a zinc compound having a deodorizing function is or are bonded or adhered thereto is contained in said granular core portion, between said granular core portion and said coating layer portion, in at least a portion of said coating layer portion or in at least a portion of a surface of said coating layer portion, or between said granular core portion and said coating layer portion, as well as in at least a portion of said coating layer portion or in at least a portion of a surface of said coating layer portion, or in at least a portion of said coating layer portion and in at least a portion of a surface of said coating layer portion.

15. A granular excrement treating article according to claim 5 characterized in that said copper compound or said zinc compound or said copper compound and said zinc compound having a deodorizing function is a water-insoluble compound.

16. A granular excrement treating article according to claim 5 characterized in that said paper powder is a mixture of a paper powder having a larger water absorbency and a paper powder having a smaller water absorbency.

17. A granular excrement treating article according to claim 5, characterized in that said powder of the organic waste is a powder of a plastic waste or a pulverized matter of an organic waste, or a mixture of these pulverized matters.

18. A granular excrement treating article according to claim 17, characterized in that said plastic waste for forming the powder of the plastic waste is a discarded plastic waste, a waste of an excrement treating article for an animal, a separation product rich in plastic waste from a waste of an excrement treating article for an animal, a paper diaper waste, a separation product rich in plastic waste from a paper diaper waste, a menstrual napkin waste, a separation product rich in plastic waste from a menstrual napkin waste, a waste of a paper diaper for an animal, a separation product rich in plastic waste from a waste of a paper diaper for an animal, a menstrual napkin waste, a separation product rich in plastic waste from a menstrual napkin waste, a waste of a menstrual napkin for an animal, a separation product rich in plastic waste from a waste of a menstrual napkin for an animal, a breast pad waste, a separation product rich in plastic waste from a breast pad waste, a sweat pad waste, a separation product rich in plastic waste from a sweat pad waste, an incontinence pad waste, a separation product rich in plastic waste from an incontinence pad waste, a waste of a sheet for an animal, a separation product rich in plastic waste from a waste of a sheet for an animal, a bed sheet waste, a separation product rich in plastic waste resulting from the classification or the like of a bed sheet waste, a mask waste, a separation product rich in plastic waste from a mask waste, an eye mask waste, a separation product rich in plastic waste from an eye mask waste, a seat head cover waste, a separation product rich in plastic waste from a seat head cover waste, a pillow cover waste, a separation product rich in plastic waste from a pillow cover waste, or a synthetic resin fiber waste, or a mixture containing two or more of them.

19. A granular excrement treating article according to claim 5, characterized in that said organic waste for forming the pulverized matter of the organic waste is a thin paper waste, a sanitary paper waste, a toilet paper waste, a tissue paper waste, a facial tissue waste, a coarse paper waste, cellulose wadding waste, a paper towel waste, a toilet seat sheet waste, a newspaper refuse, a magazine refuse, a buff powder, a mechanical pulp waste, a chemical pulp waste, a cotton-like pulp waste, a wood pulp waste, a pulverized matter of a used paper pulp, a paper powder, a fluff pulp, a waster-absorptive fiber waste, a non-woven fabric waste, a paper powder containing a water-absorptive resin, a paper powder generated upon bookbinding, a paper powder generated upon production of a non-woven fabric, a paper powder generated in a paper-making process or a paper powder generated upon production of a sanitary material, a laminate paper waste, a printing refuse of a laminate paper, an end refuse of a laminate paper, a buff powder, a corrugated cardboard refuse, a newspaper refuse, a magazine refuse, a sludge generated from the paper-making, a pulp sludge, a synthetic resin fiber waste, a wood refuse, wood shavings, a wood powder, pieces resulting from the demolition of a building or house, a waste from the new construction of a house, a paper powder, a titanium paper waste, an extraction residue of parched and milled coffee beans, used tea leaves, a vegetable refuse, used tickets or a punching refuse, or a mixture containing two or more of them.

20. A granular excrement treating article according to claim 5, characterized in that said paper powder has a particle size equal to or smaller than 0.5 mm, and is a thin paper, a thin paper waste, a sanitary paper, a sanitary paper waste, a toilet paper, a toilet paper waste, a tissue paper, a tissue paper waste, a facial tissue, a facial tissue waste, a coarse paper, a coarse paper waste, a cellulose wadding, a cellulose wadding waste, a paper towel, a paper towel waste, a toilet seat sheet waste, a newspaper, a newspaper refuse, a magazine refuse, a buff powder, a mechanical pulp, a mechanical pulp waste, a chemical pulp, a chemical pulp waste, a semi-chemical pulp, a semi-chemical pulp waste, a cotton-like pulp, a cotton-like pulp waste, a wood pulp, a wood pulp waste, a pulverized matter of a used paper pulp, a fluff pulp, a water-absorptive fiber waste, a paper powder containing a water-absorptive resin, a paper powder generated upon bookbinding, a paper powder generated upon production of a non-woven fabric, a paper powder generated in a paper-making process, or a paper powder generated upon production of a sanitary material, or a mixture of pulverized matters of two or more of them.

21. A granular excrement treating article according to claim 5, characterized in that said material having the adhesive property is a water-absorptive resin or a adhesive material, or a mixture of them.

22. A granular excrement treating article according to claim 6, characterized in that said additive includes another substance having a deodorizing effect, a substance having a sterilizing effect or a surfactant or two or more of these substances.

23. A granular excrement treating article according to claim 22, characterized in that said substance having the sterilizing effect contains a sterilizer, a fungicide, or an antiseptic agent or two or more of these agents.

24. A process for producing a material having a deodorizing function, characterized by the step of mixing an aqueous solution of a copper compound or a zinc compound or a copper compound and a zinc compound into a paper powder, thereby allowing the copper compound or the zinc compound or the copper compound and the zinc compound to be retained in the paper powder.

25. A process for producing a material having a deodorizing function, characterized by the steps of mixing an aqueous solution of a copper compound or a zinc compound or a copper compound and a zinc compound into a paper powder, and mixing the resulting mixture into an aqueous solution of a pH adjuster to form a water-insoluble complex, basic compound or hydroxide of copper or zinc or copper and zinc, thereby the formed water-insoluble basic compound or hydroxide of copper or zinc or copper and zinc to be retained in the paper powder.

26. A process for producing a material having a deodorizing function, characterized by the steps of mixing an aqueous solution of a copper compound or a zinc compound or a copper compound and a zinc compound into a paper powder including citrate into a paper powder at a temperature equal to or higher than the normal temperature, and mixing the resulting mixture into an aqueous solution of a pH adjuster to form a water-insoluble complex, basic compound or hydroxide of copper or zinc or copper and zinc, thereby the formed water-insoluble basic compound or hydroxide of copper or zinc or copper and zinc to be retained in the paper powder.

27. A process for producing a material having a deodorizing function, characterized by the steps of mixing an aqueous solution or suspension of a copper compound or a zinc compound or a copper compound and a zinc compound into carboxymethyl cellulose, thereby allowing copper or zinc ion or copper and zinc ions to be adsorbed or deposited to the carboxymethyl cellulose, and mixing the carboxymethyl cellulose having the copper or zinc ion or copper and zinc ions adsorbed thereto with a paper powder in the presence of water, thereby allowing the carboxymethyl cellulose having the copper or zinc ion or copper and zinc ions adsorbed or deposited thereto to be deposited to the paper powder.

28. A process for producing a material having a deodorizing function according to claim 24, characterized in that the paper powder is produced by classifying a pulverized matter of a sanitary goods waste having a particle size equal to or smaller than 5 mm to separate a classification product rich in plastic, and by further classifying a classification product of the pulverized matter of a paper diaper waste having a particle size equal to or smaller than 5 mm separated from the classification product rich in plastic to separate a classification product rich in a water-absorptive resin.

29. A process for producing a material having a deodorizing function according to claim 28, characterized in that the sanitary goods waste forming the pulverized matter of the sanitary goods waste is a paper diaper waste, a breast pad waste, a menstrual napkin waste or a urine-absorbing pad waste or a combination of two or more of these wastes.

30. A process for producing a material having a deodorizing function according to claim 24, characterized in that the paper powder is a pulverized matter of a paper waste, a buff powder, a pulverized matter of a corrugated cardboard refuse, a pulverized matter of a newspaper refuse, a pulverized matter of a mechanical pulp waste, a pulverized matter of a chemical pulp waste, a pulverized matter of a semi-chemical pulp waste, a pulverized matter of a wood pulp waste, a paper powder generated upon a bookbinding, a paper powder generated upon production of a non-woven fabric, a paper powder generated in a paper-making process, or a paper powder generated upon production of a sanitary paper.

31. A process for producing a material having a deodorizing function according to claim 24, characterized in that the paper powder containing the water-insoluble basic compound or hydroxide of copper or zinc or copper and zinc retained therein has a content of copper or zinc or copper and zinc equal to or higher than 0.5% by weight.

32. A process for producing a material having a deodorizing function according to claim 24, characterized in that the paper powder has a particle size equal to or smaller than 0.35 mm.

33. A process for producing a material having a deodorizing function according to claim 24, characterized in that the copper compound or the zinc compound or the copper compound and the zinc compound retained in the paper powder is a water-insoluble inorganic compound of copper or zinc or copper and zinc.

34. A process for producing a material having a deodorizing function according to claim 24, characterized in that the hydroxide or basic compound of copper or zinc or copper and zinc retained in the paper powder is water-insoluble.

35. A process for producing a granular excrement treating article, characterized by the steps of mixing a powder of an organic waste having a particle size equal to or smaller than 5 mm with a material having an adhesive property in an amount smaller than that of said powder of the organic waste, granulating the resulting mixture to form a granulated matter having a grain size equal to or larger than 1 mm, coating a copper compound or a zinc compound or a copper compound and a zinc compound having a deodorizing function to at least a portion of a surface of the formed granulated matter, thereby, as a core portion, forming the granulated matter coated with the copper compound or the zinc compound or the copper compound and the zinc compound, coating a coating composition containing a paper powder and a material having an adhesive property to at least a portion of a surface of said core portion to form a coated granulated matter, and drying said coated granulated matter to provide a dried granular material having a water content equal to or lower than 12% by weight.

36. A process for producing a granular excrement treating article, characterized by the steps of mixing, with a powder of an organic waste having a particle size equal to or smaller than 5 mm, an amount of a material having an adhesive property smaller than that of said powder of the organic waste and an additive, granulating the resulting mixture to form a granulated matter having a grain size equal to or larger than 1 mm, coating a copper compound or a zinc compound or a copper compound and a zinc compound having a deodorizing function to at least a portion of a surface of the formed granulated matter, thereby, as a core portion, forming the granulated matter coated with the copper compound or the zinc compound or the copper compound and the zinc compound, coating a coating composition containing a paper powder and a material having an adhesive property to at least a portion of a surface of said core portion to form a coated granulated matter, and drying said coated granulated matter to provide a dried granular material having a water content equal to or lower than 12% by weight.

37. A process for producing a granular excrement treating article, characterized by the steps of mixing a powder of an organic waste having a particle size equal to or smaller than 5 mm with a material having an adhesive property in an amount smaller than that of said powder of the organic waste, granulating the resulting mixture to form a granulated matter, as a core portion, having a grain size equal to or larger than 1 mm, coating a coating composition containing a paper powder and a material having an adhesive property to at least a portion of a surface of said core portion to form a coated granulated matter coated with said coating composition, coating a copper compound or a zinc compound or a copper compound and a zinc compound having a deodorizing function to at least a portion of a surface of the coated granulated matter to form a coated granulated matter coated with said copper compound or said zinc compound or said copper compound and said zinc compound, and drying said coated granulated matter coated with said copper compound or said zinc compound or said copper compound and said zinc compound to provide a dried granular material having a water content equal to or lower than 12% by weight.

38. A process for producing a granular excrement treating article, characterized by the steps of mixing a powder of an organic waste having a particle size equal to or smaller than 5 mm with a material having an adhesive property in an amount smaller than that of said powder of the organic waste and an additive, granulating the resulting mixture to form a granulated matter, as a core portion, having a grain size equal to or larger than 1 mm, coating a coating composition containing a paper powder and a material having an adhesive property to at least a portion of a surface of said core portion to form a coated granulated matter coated with the coating composition, coating a copper compound or a zinc compound or a copper compound and a zinc compound having a deodorizing function to at least a portion of a surface of said core portion to form a coated granulated matter coated with the copper compound or the zinc compound or the copper compound and the zinc compound, and drying said coated granulated matter coated with the copper compound or the zinc compound or the copper compound and the zinc compound to provide a dried granular material having a water content equal to or lower than 12% by weight.

39. A process for producing a granular excrement treating article, characterized by the steps of mixing a powder of an organic waste having a particle size equal to or smaller than 5 mm with a material having an adhesive property in an amount smaller than that of said powder of the organic waste, granulating the resulting mixture to form a granulated matter, as a core portion, having a grain size equal to or larger than 1 mm, coating a coating composition containing a paper powder retaining a copper compound or a zinc compound or a copper compound and a zinc compound having a deodorizing function therein and a material having an adhesive property to at least a portion of a surface of said core portion to form a coated granulated matter with at least a portion of the surface of said granular core portion coated with said coating composition, and drying said coated granulated matter to provide a dried granular material having a water content equal to or lower than 12% by weight.

40. A process for producing a granular excrement treating article, characterized by the steps of mixing a powder of an organic waste having a particle size equal to or smaller than 5 mm with a material having an adhesive property in an amount smaller than that of said powder of the organic waste, granulating the resulting mixture to form a granulated matter, as a core portion, having a grain size equal to or larger than 1 mm, coating a coating composition containing a paper powder retaining a copper compound or a zinc compound or a copper compound and a zinc compound having a deodorizing function therein and a material having an adhesive property to at least a portion of a surface of said core portion to form a coated granulated matter with at least a portion of the surface of said granular core portion coated with said coating composition, and drying said coated granulated matter to provide a dried granular material having a water content equal to or lower than 12% by weight.

41. A process for producing a granular excrement treating article, characterized by the steps of mixing a powder of an organic waste having a particle size equal to or smaller than 5 mm with a material having an adhesive property in an amount smaller than that of said powder of the organic waste and an additive, granulating the resulting mixture to form a granulated matter, as a core portion, having a grain size equal to or larger than 1 mm, coating a coating composition containing a paper powder retaining a copper compound or a zinc compound or a copper compound and a zinc compound having a deodorizing function therein and a material having an adhesive property to at least a portion of a surface of said core portion to form a coated granulated matter with at least a portion of the surface of said granular core portion coated with said coating composition, and drying said coated granulated matter to provide a dried granular material having a water content equal to or lower than 12% by weight.

42. A process for producing a granular excrement treating article, characterized by the steps of mixing a powder of an organic waste having a particle size equal to or smaller than 5 mm with a material having an adhesive property in an amount smaller than that of said powder of the organic waste and an additive, granulating the resulting mixture to form a granulated matter, as a core portion, having a grain size equal to or larger than 1 mm, coating a coating composition containing a paper powder retaining a copper compound or a zinc compound or a copper compound and a zinc compound having a deodorizing function therein, a material having an adhesive property and an additive to at least a portion of a surface of said core portion to form a coated granulated matter with at least a portion of the surface of said granular core portion coated with said coating composition, and drying said coated granulated matter to provide a dried granular material having a water content equal to or lower than 12% by weight.

43. A process for producing a granular excrement treating article according to claim 35, characterized in that the material having the adhesive property is a water-absorptive resin or an adhesive material or a mixture of them.

44. A process for producing a granular excrement treating article according to claim 35, characterized in that the powder of the organic waste is a powder of a plastic waste or a pulverized matter of an organic waste, or a mixture of a pulverized matter of a plastic waste and a pulverized matter of an organic waste.

45. A process for producing a granular excrement treating article according to claim 44, characterized in that the plastic for forming the powder of the plastic or the pulverized matter of the plastic waste is a plastic, a discarded plastic, a waste of an excrement treating article for an animal, a separation product rich in plastic separated from a waste of an excrement treating article for an animal, a paper diaper waste, a separation product rich in plastic separated from a paper diaper waste, a menstrual napkin waste, a separation product rich in plastic separated from a menstrual napkin waste, a waste of a paper diaper for an animal, a separation product rich in plastic separated from a waste of a paper diaper for an animal, a menstrual napkin waste, a separation product rich in plastic separated from a menstrual napkin waste, a waste of a menstrual napkin for an animal, a separation product rich in plastic separated from a waste of a menstrual napkin for an animal, a breast pad waste, a separation product rich in plastic separated from a breast pad waste, a sweat pad waste, a separation product rich in plastic separated from a sweat pad waste, an incontinence pad waste, a separation product rich in plastic separated from an incontinence pad waste, a waste of a sheet for an animal, a separation product rich in plastic separated from a waste of a sheet for an animal, a bed sheet waste, a separation product rich in plastic separated from a bed sheet waste, a mask waste, a separation product rich in plastic separated from a mask waste, an eye mask waste, a separation product rich in plastic separated from an eye mask waste, a seat head cover waste, a separation product rich in plastic separated from a seat head cover waste, a pillow cover waste, a separation product rich in plastic separated from a pillow cover waste, or a synthetic resin fiber waste, or a mixture containing two or more of them.

46. A process for producing a granular excrement treating article according to claim 44, characterized in that the organic waste forming the powder of the organic waste or the pulverized matter of the organic waste is a thin paper waste, a sanitary paper waste, a waste of an excrement treating article for an animal, a bed sheet waste, a mask waste, an eye mask waste, a seat head cover waste, a pillow cover waste, a paper diaper waste, a menstrual napkin waste, a waste of a paper diaper for an animal, a menstrual napkin waste, a waste of a menstrual napkin for an animal, a breast pad waste, a sweat pad waste, an incontinence pad waste, a waste of a sheet for an animal, a toilet paper waste, a tissue paper waste, a facial tissue waste, a coarse paper waste, a cellulose wadding waste, a paper towel waste, a toilet seat sheet waste, a newspaper refuse, a magazine refuse, a buff powder, a mechanical pulp waste, a chemical pulp waste, a semi-chemical pulp waste, a cotton-like pulp waste, a wood pulp waste, a pulverized matter of a used paper pulp, a paper powder, a fluff pulp, a water-absorptive fiber waste, a non-woven fabric, a non-woven fabric waste, a paper powder containing a water-absorptive resin, a paper powder generated upon bookbinding, a paper powder generated upon production of a non-woven fabric, a paper powder generated in a paper-making process, or a paper powder generated upon production of a sanitary material, a laminate paper waste, a printing refuse of a laminate paper, an end refuse of a laminate paper, a buff powder, a corrugated cardboard waste, newspaper refuse, a magazine refuse, a paper-making sludge, a pulp sludge, a non-woven fabric refuse, a synthetic resin fiber waste, a wood refuse, wood shavings, a wood powder, pieces resulting from the demolition of a building or house, a waste from the new construction of a house, a paper powder, a titanium paper waste, an extraction residue of parched and milled coffee beans, used tea leaves, a vegetable refuse, used tickets or a punching refuse, or a mixture of two or more of them.

47. A process for producing a granular excrement treating article according to claim 35, characterized in that the paper powder is a thin paper, a thin paper waste, a sanitary paper, a sanitary paper waste, a toilet paper, a toilet paper waste, a tissue paper, a tissue paper waste, a facial tissue, a facial tissue waste, a coarse paper, a coarse paper waste, a cellulose wadding, a cellulose wadding waste, a paper towel, a paper towel waste, a toilet seat sheet waste, a newspaper, a newspaper refuse, a magazine refuse, a buff powder, a mechanical pulp, a mechanical pulp waste, a chemical pulp, a chemical pulp waste, a semi-chemical pulp, a semi-chemical pulp waste, a cotton-like pulp, a cotton-like pulp waste, a wood pulp, a wood pulp waste, a pulverized matter of a used paper pulp, a paper powder, a fluff pulp, a water-absorptive fiber waste, a paper powder containing a water-absorptive resin, a paper powder generated upon bookbinding, a paper powder generated upon production of a non-woven fabric, a paper powder generated in a paper-making process, or a paper powder generated upon production of a sanitary material, or a mixture of two or more of the pulverized matters, and said paper powder has a particle size equal to or smaller than 0.5 mm.

48. A process for producing a granular excrement treating article according to claim 36, characterized in that the additive includes another substance having a deodorizing effect, a substance having sterilizing effect, a surfactant or a coloring substance, or two or more of these substances.

49. A process for producing a granular excrement treating article according to claim 37, characterized in that the substance having the sterilizing effect contains a sterilizer, a fungicide or an antiseptic agent, or two or more of these agents.
